(19) United States
(12) Patent Application Publication
(10) Pub. No.: US 2019/0350955 A1
(43) Pub. Date: Nov. 21, 2019

(54) METHODS AND PHARMACEUTICAL COMPOSITIONS FOR THE TREATMENT OF CARDIOMYOPATHIES

(71) Applicants: INSERM (Institut National de la Sante et de la Recherche Medicale, Paris (FR); Centre National de la Recherche Scientifique (CNRS), Paris (FR); Association Institut de Myologie; Sorbonne Universite, Paris (FR)

(72) Inventors: Nicolas VIGNIER, Paris Cedex 13 (FR); Mathias MERICSKAY, Paris (FR); Zhenlin LI, Paris (FR); Antoine MUCHIR, Paris Cedex 13 (FR); Gisele BONNE, Paris Cedex 13 (FR); Nicolas DIGUET, Paris (FR)

(21) Appl. No.: 16/313,332

(22) PCT Filed: Jun. 29, 2017
(86) PCT No.: PCT/EP2017/066118

§ 371 Date: Dec. 26, 2018

(30) Foreign Application Priority Data
Jun. 30, 2016 (EP) 16305804.3

(51) Int. Cl. A61K 31/706; A61K 45/06; A61P 9/10

(52) U.S. Cl. CPC A61K 31/706; A61K 9/0053; A61P 9/10; A61K 45/06

(57) ABSTRACT

The present invention relates to methods and pharmaceutical compositions for the treatment of cardiomyopathies. The inventors showed that the nicotinamide riboside kinase Nmrk2 gene involved in NAD+ biosynthetic pathway is strongly induced in the heart of the mouse models of dilated cardiomyopathy and that Nmrk2 is an AMPK and PPARα responsive gene. They also showed that the NMRK enzymes substrate nicotinamide riboside supplementation in food markedly improves cardiac functions and reduces eccentric remodeling. The inventors demonstrated that both the NMRK1 and NMRK2 protein are expressed in the human healthy heart, that NMRK2 protein level is increased in human failing hearts as it is the case in mouse failing hearts in several models of heart failure and cardiomyopathies. In particular, the present invention relates to nicotinamide riboside for use in the treatment of cardiomyopathy in a human subject in need thereof.

### FIELD OF THE INVENTION

The present invention relates to methods and pharmaceutical compositions for the treatment of cardiomyopathies.

### BACKGROUND OF THE INVENTION

Cardiomyopathies are heart muscle disorders which represent a heterogeneous group of diseases that often lead to progressive heart failure with significant morbidity and mortality. Common symptoms include dyspnea and peripheral oedema, and risks of having dangerous forms of irregular heart rate and sudden cardiac death are increased. The most common form of cardiomyopathy is dilated cardiomyopathy. Dilated cardiomyopathy is a heart muscle disorder characterized by dilatation and systolic dysfunction of the left or both ventricles (Elliott, P., Andersson, B., Arbustini .E., Bilinska, Z., Cecchi, F., Charron, P., Dubourg, O., Ktihl, U., Maisch, B., McKenna, W. J., et al. (2008) Classification of the cardiomyopathies: a position statement from the european society of cardiology working group on myocardial and pericardial diseases. Eur. Heart J., 29, 270-276). The ventricular walls become thin and stretched, compromising cardiac contractility and ultimately resulting in poor left ventricular function.

Studies have indicated that nicotinamide adenine dinucleotide (NAD+) content is a critical determinant for heart function and structure (Hsu, C.-P., Oka, S., Shao, D., Hariharan, N. and Sadoshima, J. (2009) Nicotinamide Phosphoribosyltransferase Regulates Cell Survival Through NAD+ Synthesis in Cardiac Myocytes. Circ. Res., 105, 481-49) (Pillai, V. B., Sundaresan, N. R., Kim, G., Gupta, M., Rajamohan, S. B., Pillai, J. B., Samant, S., Ravindra, P. V., Isbatan, A. and Gupta, M. P. (2010) Exogenous NAD blocks cardiac hypertrophic response via activation of the SIRT3-LKB1-AMP-activated kinase pathway. J. Biol. Chem., 285, 3133-3144). More particularly, alteration of NAD+ homeostasis in the failing heart has been observed in several models of heart failure (Mericskay, M. (2016). Nicotinamide adenine dinucleotide homeostasis and signaling in heart disease: Pathophysiological implications and therapeutic potential. Arch Cardiovasc Dis 109, 207-215).

Nicotinamide adenine dinucleotide is a metabolic co-factor that is present in cells either in its oxidized (NAD+) or reduced (NADH) form. NAD+ first described as a coenzyme of redox reactions, is an important player in metabolism. As co-substrate it participates to glycolysis, lactate metabolism, tricarboxylic acid (TCA) cycle, and electron transfer chain. The roles of NAD+ have expanded beyond its role as a coenzyme, as NAD+ also acts as degradation substrate for a wide range of enzymes, such as sirtuins (Imai, S., Armstrong, C. M., Kaeberlein, M. and Guarente, L. (2000) Transcriptional silencing and longevity protein Sir2 is an NAD-dependent histone deacetylase. Nature, 403, 795-800) and poly(ADP-ribose) polymerase (PARPs) (Shimizu, Y., Hasegawa, S., Fujimura, S. and Sugimura, T. (1967) Solubilization of enzyme forming ADPR polymer from NAD. Biochem. Biophys. Res. Commun., 29, 80-83). Among the 17 isoforms of PARPs a vast majority are nuclear enzymes of eukaryotic cells that participate in DNA repair in response to genotoxic stress. PARP-1 and PARP-2 represent around 95% of PARPs activity and when activated by DNA single-strand breaks, PARPs initiate an energy-consuming cycle by transferring ADP-ribose units from NAD+ to nuclear proteins (Bai, P. (2015) Biology of Poly(ADP-Ribose) Polymerases: The Factotums of Cell Maintenance. Mol. Cell, 58, 947-958) This process results in rapid depletion of the intracellular NAD+ and ATP pools. While in conditions of physiological equilibrium the consumption of NAD+ by these pathways is counterbalanced by dietary sources of tryptophan (TRP) and vitamins B3 (niacins) precursors, induction of NAD+ consuming activities could result in insufficiencies within the NAD+ metabolome in a pathological condition (Bogan, K. L., and Brenner, C. (2008). Nicotinic acid, nicotinamide, and nicotinamide riboside: a molecular evaluation of NAD+ precursor vitamins in human nutrition. Annu Rev Nutr 28, 115-130). Alternatively, the depletion in cellular NAD+ levels could be due to a repression of one or several enzymes involved in NAD+ synthesis. As previously mentioned, alteration of NAD+ homeostasis has been observed in several model of heart failure.

Despite current strategies to aggressively manage cardiomyopathy, the disorder remains a common cause of heart failure and a prevalent diagnosis in individuals referred for cardiac transplantation. There is a need for new evidence-based and cost-effective treatments.

### SUMMARY OF THE INVENTION

The present invention relates to methods and pharmaceutical compositions for the treatment of cardiomyopathies. In particular, the invention is defined by the claims.

### DETAILED DESCRIPTION OF THE INVENTION

Surprisingly, the inventors showed that the nicotinamide riboside kinase Nmrk2 involved in NAD+ biosynthetic pathway, is strongly induced in the heart of the mouse models of dilated cardiomyopathy and that Nmrk2 is an AMPK and PPARα responsive gene. They also showed that nicotinamide riboside supplementation in food markedly improves cardiac functions and reduces eccentric remodeling. Nicotinamide riboside is a precursor of NAD+ that is phosphorylated by the nicotinamide riboside kinases NMRK1 and NMRK2 to generate nicotinamide mononucleotide (NMN), which is then converted to NAD+ by nicotinamide/nicotinate adenylyltransferases NMNAT1, NMNAT2 and NMNAT3. Nicotinamide riboside can also be converted by several cellular enzymes into nicotinamide, which is used by the nicotinamide phosphoribosyltransferase NAMPT to generate NMN, which is then converted to NAD+ by NMNAT enzymes. The inventors also showed that both the NMRK1 and NMRK2 protein are expressed in the human healthy heart, that NMRK2 protein level is increased in human failing hearts as it is the case in mouse failing hearts in several models of heart failure, suggesting that nicotinamide riboside could be a favoured precursor for NAD+ synthesis in the human failing heart.

A first aspect of the present invention relates to a method of treating cardiomyopathy in a subject in need thereof, comprising administering to the subject a therapeutically effective amount of nicotinamide riboside.

A further aspect of the present invention relates to a method of treating heart failure in a subject in need thereof, comprising administering to the subject a therapeutically effective amount of nicotinamide riboside

As used herein, the term “subject” denotes a mammal, such as a rodent, a feline, a canine, and a primate. Preferably, a subject according to the invention is a human.

As used herein, the term “heart failure” refers to a progressive, debilitating condition wherein the heart loses its ability to function as a circulatory pump.

As used herein, the term “cardiomyopathy” has its general meaning in the art and refers to any disease of the heart muscle. Cardiomyopathy can be acquired or inherited. Common symptoms include dyspnea and peripheral oedema, and risks of having dangerous forms of irregular heart rate and sudden cardiac death are increased. Cardiomyopathy often leads to progressive heart failure, i.e the incapacity of the cardiac pump to maintain sufficient blood flow to meet the basal bodily needs for oxygen. The main types of cardiomyopathy include dilated cardiomyopathy, hypertrophic cardiomyopathy, non-obstructive cardiomyopathy, restrictive cardiomyopathy, left ventricular non-compaction, arrhythmogenic right ventricular cardiomyopathy. Dilated cardiomyopathy is characterized by dilatation and systolic dysfunction of the left or both ventricles. The ventricular walls become thin and stretched, compromising cardiac contractility and ultimately resulting in poor left ventricular function. In one embodiment, the cardiomyopathy is dilated cardiomyopathy. In one embodiment, the cardiomyopathy is rare cardiomyopathy.

In one embodiment, the rare cardiomyopathy is one life-threatening symptom among several in complex disorders and is selected but not restricted from the group consisting of Congenital cardiomyopathy, Emery Dreiffuss Muscular Dystrophy, Duchenne and Becker Muscular Dystrophy, Limb-Girdle dystrophy, Steinert disease, Danon disease, Myofibrillar Myopathy, Arrhythmogenic dysplasia, Peripartum cardiomyopathy, Tako Tsubo cardiomyopathy, Nemaline Myopathies or RASopathies.

In one embodiment, the cardiomyopathy may derive from the following non familial causes: obesity, infants of diabetic mothers, athletic training, amyloid (al/prealbumin), myocarditis (infective/toxic/immune), Kawasaki disease, eosinophilic (churg strauss, syndrome), viral persistence, pregnancy, endocrine, nutritional (thiamine, carnitine, selenium, hypophosphataemia, hypocalcaemia), alcohol, tachycardiomyopathy, inflammation, amyloid (AL/prealbumin), scleroderma, endomyocardial fibrosis, hypereosinophilic syndrome, drugs (serotonin, methysergide ergotamine, mercurial agents, busulfan), carcinoid heart disease, metastatic cancers, antineoplastic drugs (anthracyclines, antimetabolites; alkylating agents;

taxol, hypomethylating agent, monoclonal antibodies, tyrosine kinase inhibitors, immunomodulating agents), psychiatric drugs (clozapine, olanzapine; chlorpromazine, risperidone, lithium, methylphenidate, tricyclic antidepressants) and other drugs such as chloroquine, all-trans retinoic acid, antiretroviral agents and phenothiazines.

In one embodiment, the rare cardiomyopathy may derive from mutations or rare variants of one or several of the following genes (but it is not limited to these genes) as listed by their official gene symbol provided by the HUGO Gene Nomenclature Committee and, in brackets, their chromosomic localization in the human genome: A2ML1 (12p13.31). AARS2 (6p21.1), ABCC9 (12p12.1), ACAD9 (3q21.3), ACADVL (17p13.1), ACTA1 (1q42.13), ACTC1 (15q14), ACTN2 (1q43), AGK (7q34), AGL (1p21.2), AGPAT2 (9q34.3), ALMS1 (2p13.1), ANK2 (4q25-26), ANKRD1(10q23.31), ANO5 (11p14.3), ATP5E (20q13.32), ATPAF2 (17p11.2), BAG3 (10q26.11), BRAF (7q34), BSCL2 (11q12.3), CALR3 (19p13.11), CASQ2 (1p13.1), CAV3 (3p25.3), CAVIN4 (9q31.1), CHRM2 (7q33), COA5 (2q11.2), COA6 (1q42.2), COL7A1 (3p21.31), COQ2 (4q21.22-21.23), COX15 (10q24.2), COX6B1 (19q13.12), CRYAB (11q23.1), CSRP3 (11p15.1), CTNNA3 (10q21.3), CTNNB1 (3p22.1), DES (2q35), DLD (7q31.1), DMD (Xp21.2-21.1), DNAJC19 (3q26.33), DNM1L (12p11.21), DOLK (9q34.11), DSC2 (18q12.1), DSG2 (18q12.1), DSP (6p24.3), DTNA (18q12.1), ELAC2 (17p12), EMD (Xq28), EYA4 (6q23.2), FAH (15q25.1), FHL1 (Xq26.3), FHL2 (2q12.2), FHOD3 (18q12.2), FKRP (19q13.32), FKTN (9q31.2), FLNC (7q32.1), FOXD4 (9p24.3), FOXRED1 (11q24.2), FXN (9q21.11), GAA (17q25.3), GATA4 (8p23.1), GATA5 (20q13.33), GATA6 (18g11.2), GATAD1 (7q21.2), GFM1 (3q25.32), GLA (Xq22.1), GLB1 (3p22.3), GNPTAB (12q23.2), GUSB (7q11.21), HCN4 (15q24.1), HFE (6p22.2), HRAS (11p15.5), IDH2 (15q26.1), ILK (11p15.4), JPH2 (20q13.12), JUP (17q21.2), KCNH2 (7q36.1), KCNJ2 (17q24.3), KCNJ8 (12p12.1), KCNQ1 (11p15.5-15.4), KLF10 (8q22.3), KRAS (12p12.1), LAMA2 (6q22.33), LAMA4 (6q21), LAMP2 (Xq24), LDB3 (10q23.2), LIAS (4p14), LMNA (1q22), LZTR1 (22q11.21), MAP2K1 (15q22.31), MAP2K2 (19p13.3), MIB1 (18q11.2), MLYCD (16q23.3), MRPL3 (3q22.1), MRPL44 (2q36.1), MRPS22 (3q23), MTO1(6q13), MYBPC3 (11p11.2), MYH6 (14q11.2), MYH7 (14q11.2), MYL2 (12q24.11), MYL3 (3p21.31), MYLK2 (20q11.21), MYOM1 (18p11.31), MYOT (5q31.2), MYOZ2 (4q26), MYPN (10q21.3), NEBL (10p12.31), NEXN (1p31.1), NF1 (17q11.2), NKX2-5 (5q35.1), NNT (5p12), NOTCH1 (9q34.3), NRAS (1p13.2), OBSCN (1q42.13), OBSL1 (2q35), OPA3 (19q13.32), PDHA1 (Xp22.12), PDLIM3 (4q35.1), PERP (6q23.3), PHKA1 (Xq13.1), PKP2 (12p11.21), PKP4 (2q24.1), PLN (6q22.31), PMM2 (16p13.2), PPP1R13L (19q13.32), PRDM16 (1p36.32), PRKAG2 (7q36.1), PSEN1 (14q24.2), PSEN2 (1q42.13), PTPN11 (12q24.13), RAF1 (3p25.2), RASA2 (3q23), RBM20 (10q25.2), RIT1 (1q22), RRAS (19q13.33), RYR2 (1q43), SCNSA (3p22.2), SCO2 (22q13.33), SDHA (5p15.33), SGCA (17q21.33), SGCB (4q12), SGCD (5q33.2-33.3), SHOC2 (10q25.2), SLC22A5 (5q31.1), SLC25A3 (12q23.1), SLC25A4 (4q35.1), SOS1 (2p22.1), SOS2 (14q21.3), SPEG (2q35), SPRED1 (15q14), SURF1 (9q34.2), SYNE1 (6q25.2), SYNE2 (14q23.2), TAZ (Xq28), TBX20 (7p14.2), TCAP (17q12), TGFB3 (14q24.3), TMEM43 (3p25.1), TMEM70 (8q21.11), TMPO (12q23.1), TNNC1 (3p21.1), TNNI3 (19q13.42), TNNI3K (1p31.1), TNNT2 (1q32.1), TOR1AIP1 (1q25.2), TPM1 (15q22.2), TRIM63 (1p36.11), TSFM (12q14.1), TTN (2q31.2). TTR (18q 12.1), TXNRD2 (22q11.21), VCL (10q22.2), XK (Xp21.1).

In one embodiment, the rare cardiomyopathy is associated with NAD+ deficiency in the myocardium and may derive from reduced expression level or reduced activity of the proteins encoded by the following genes or mutations in one or several of the following genes involved in NAD+ biosynthesis or in regulation of one of those genes (but it is not limited to these genes): NAMPT (7q22.3), NMRK1 (9q21.13), NMRK2 (19p13.3), NMNAT1 (1p36.22), NMNAT2 (1q25.3), NMNAT3 (3q23), NADSYN1 (11q13.4), NAPRT (8q24.3), TDO2 (4q32.1), AFMID (17q25.3), KMO (1q43), KYNU (2q22.2), HAAO (2p21), QPRT (16p11.2), NT5E (6q14.3), SRF (6p21.1), MKL1 (22q13 or 1-q13.2 in case of translocation), MKL2 (16p13.12), CKM (19q13.32).

In one embodiment, the rare cardiomyopathy is associated with NAD+ deficiency in the myocardium and may derive from increased expression level or increased activity of the proteins encoded by the following genes or mutations in one or several of the following genes involved in NAD+ biosynthesis (but it is not limited to these genes): PARP1 (1q42.12), PARP2 (14q11.2), CD38 (4p15.32), BST1 (4p15.32), ART1 (11p15.4).

Thus, in one embodiment, the rare cardiomyopathy derives from mutations or rare variant of one or several genes selected from the group consisting of NAMPT, NMRK1, NMRK2, NMNAT, NMNAT2, NMNAT3, NADSYN1, NAPRT, TDO2, AFMID, KMO, KYNU, HAAO, QPRT, NTSE, SRF, MKL1, MKL2 or CKM.

Thus, in one embodiment, the rare cardiomyopathy derives from mutations or rare variant of one or several genes selected from the group consisting of PARP1, PARP2, CD38, BST1, ART1.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The term “treatment” encompasses the prophylactic treatment. As used herein, the term “prevent” refers to the reduction in the risk of acquiring or developing a given condition, or the reduction or inhibition of the recurrence or said condition in a subject who is not ill, but who has been or may be near a subject with the disease.

As used herein, the term “nicotinamide riboside” (NR) has its general meaning in the art and refers to a pyridine-nucleoside form of vitamin B3 that is a precursor to nicotinamide adenine dinucleotide or NAD+. Molecular formula of nicotinamide riboside is C₁₁H₁₅N₂O₅⁺. The term “nicotinamide riboside” includes any derivative of nicotinamide riboside.

Nicotinamide riboside formula (I):

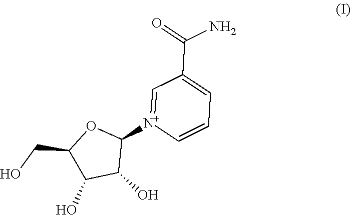 [SMILES: NC(=O)c1ccc[n+]([C@@H]2O[C@H](CO)[C@@H](O)[C@H]2O)c1]

In some embodiments, nicotinamide riboside is administrated in combination with a therapeutically effective amount of AMPK activator or/and a therapeutically effective amount of PPARα agonist.

As used herein, the term “AMPK” (5′ adenosine monophosphate-activated protein kinase) has its general meaning in the art and refers to AMP-activated protein kinase. AMPK is an energy sensor protein kinase that plays a key role in regulating cellular energy metabolism. In response to reduction of intracellular ATP levels, AMPK activates energy-producing pathways and inhibits energy-consuming processes. AMPK acts via direct phosphorylation of metabolic enzymes, and by longer-term effects via phosphorylation of transcription regulators.

As used herein, the term “AMPK activator” has its general meaning in the art and refers to any compound, as well as its derivatives and prodrugs, that promotes activity of AMPK directly, or indirectly (for example, any compound that increases intracellular AMP concentration is an AMPK activator) or to any compound that enhances AMPK gene expression,

As used herein, the term “PPARα” has its general meaning in the art and refers to Peroxisome proliferator-activated receptor alpha, also known as NR1C1 (nuclear receptor subfamily 1, group C, member 1). PPARα is a nuclear receptor protein and is a key regulator of lipid metabolism.

As used herein, the term “PPARα agonist” refers to any compound, as well as its derivatives and prodrugs, natural or not, that is able to bind to PPARα and promotes PPARα activity or to any compound that enhances PPARα gene expression.

In some embodiments, the AMPK activator or/and the PPARα agonist is a small organic molecule. The term “small organic molecule” refers to a molecule of a size comparable to those organic molecules generally used in pharmaceuticals. The term excludes biological macromolecules (e. g., proteins, nucleic acids, etc.). Preferred small organic molecules range in size up to about 5000 Da, more in particular up to 2000 Da, and most in particular up to about 1000 Da.

In one embodiment, the AMPK activator according to the invention is thienopyridone derivatives. In one embodiment, the AMPK activator according to the invention is imidazole derivatives. In one embodiment, the AMPK activator according to the invention is furanothiazolidine derivatives. In one embodiment, the AMPK activator according to the invention is metformin. In one embodiment, the AMPK activator according to the invention is troglitazone. In one embodiment, the AMPK activator according to the invention is phenformin. In one embodiment, the AMPK activator according to the invention is galegine. In one embodiment, the AMPK activator according to the invention is resveratrol. In one embodiment, the AMPK activator according to the invention is berberine. In one embodiment, the AMPK activator according to the invention is arctigenin. In one embodiment, the AMPK activator according to the invention is 5-Aminoimidazole-4-carboxamide ribonucleotide (AICAR). In one embodiment, the AMPK activator according to the invention is C13. In one embodiment, the AMPK activator according to the invention is antifolate drug. In one embodiment, the AMPK activator according to the invention is methotrexate. In one embodiment, the AMPK activator according to the invention is pemetrexed. In one embodiment, the AMPK activator according to the invention is A-592107. In one embodiment, the AMPK activator according to the invention is A-769662. In one embodiment, the AMPK activator according to the invention is cyclic benzimidazole derivatives. In one embodiment, the AMPK activator according to the invention is pyrrolo [3,2-b]pyridines. In one embodiment, the AMPK activator according to the invention is pyrrolo [2,3-d]pyrimidine derivatives. In one embodiment, the AMPK activator according to the invention is alkene oxindole derivatives. In one embodiment, the AMPK activator according to the invention is spirocyclic indolinone derivatives. In one embodiment, the AMPK activator according to the invention is 3,3-dimethyl tetrahydroquinoline derivatives. In one embodiment, the AMPK activator according to the invention is thieno [2,3-b]pyridinediones.

Examples of AMPK activator are described in the following patent applications: WO2014128549, WO2006001278, EP1907369, WO2007019914, WO2009124636, WO2007002461, WO2013153479.

In some embodiments, the PPARα agonist is fibrate drugs. In some embodiments, the PPARα agonist is clofibrate. In some embodiments, the PPARα agonist is gemfibrozil. In some embodiments, the PPARα agonist is ciprofibrate. In some embodiments, the PPARα agonist is fenofibrate. In some embodiments, the PPARα agonist is bezafibrate. In some embodiments, the PPARα agonist is GW7647. In some embodiments, the PPARα agonist is GW501516. In some embodiments, the PPARα agonist is Wy 14,643. In some embodiments, the PPARα agonist is rosiglitazone. In some embodiments, the PPARα agonist is pioglitazone. In some embodiments, the PPARα agonist is glitazars. In some embodiments, the PPARα agonist is aleglitazar. In some embodiments, the PPARα agonist is tesaglitazar. In some embodiments, the PPARα agonist is ragaglitazar. In some embodiments, the PPARα agonist is muraglitazar. In some embodiments, the PPARα agonist is KRP-297. In some embodiments, the PPARα agonist is GW-409544. In some embodiments, the PPARα agonist is Ly-510929.

Examples of PPARα agonist are described in the following patent applications: WO02092084, WO2004005266, WO2004020420, WO2004041275, WO2008031500, WO2010071813

Examples of PPARα agonist are described in James E. Klaunig, Michael A. Babich, Karl P. Baetcke, Jon C. Cook, J. Chris Corton, Raymond M. David, John G. DeLuca, David Y. Lai. Richard H. McKee, Jeffrey M. Peters, Ruth A. Roberts & Penelope A. Fenner-Crisp (2003) PPARα Agonist-Induced Rodent Tumors: Modes of Action and Human Relevance, Critical Reviews in Toxicology, 33:6, 655-780.

In one embodiment, nicotinamide riboside, the AMPK activator or/and the PPARα agonist are administered simultaneously, at essentially the same time, or sequentially.

In some embodiments, nicotinamide riboside or/and the AMPK activator or/and the PPARα agonist of the invention is administered to the subject with a therapeutically effective amount.

The terms “administer” or “administration” refer to the act of injecting or otherwise physically delivering a substance as it exists outside the body (e.g., nicotinamide riboside or/and AMPK activator or/and PPARα agonist of the present invention) into the subject, such as by oral, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of nicotinamide riboside or/and AMPK activator or/and PPARα agonist for the treatment of cardiomyopathy at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration. route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250, 500 and 1000 mg of the active ingredient for the symptomatic adjustment of the dosage to the subject to be treated. A medicament typically contains from about 0.01 mg to about 1000 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The compositions according to the invention are formulated for parenteral, transdermal, oral, rectal, intrapulmonary', subcutaneous, sublingual, topical or intranasal administration.

Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

In one embodiment, the compositions according to the invention are formulated for oral administration.

In one embodiment, nicotinamide riboside is administered orally. In one embodiment, nicotinamide riboside is administered as dietary supplement. For instance, nicotinamide riboside is marketed by Chromadex (Niageng).

In one embodiment, the compositions according to the invention are formulated for parental administration. The pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Typically the active ingredient of the present invention (i.e. nicotinamide riboside or/and AMPK activator or/and PPARα agonist) is combined with pharmaceutically acceptable excipients, and optionally sustained-release matrices, such as biodegradable polymers, to form pharmaceutical compositions.

The term “pharmaceutically” or “pharmaceutically acceptable” refers to molecular entities and compositions that do not produce an adverse, allergic or other untoward reaction when administered to a mammal, especially a human, as appropriate.

A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports.

In some embodiments, nicotinamide riboside or/and the AMPK activator or/and the PPARα agonist of the present invention is administered to the subject in combination with an active ingredient.

In some embodiments, nicotinamide riboside or/and the AMPK activator or/and the PPARα agonist of the present invention is administered to the subject in combination with a standard treatment of cardiomyopathy.

The invention will be further illustrated by the following figures and examples. However, these examples and figures should not be interpreted in any way as limiting the scope of the present invention.

### FIGURES

FIG. 1: NAD+ salvage pathway is impaired in DIVA cardiomyopathy.

(a) Quantification of cellular NAD+ content (pmol/mg tissue) in heart from Lmna^{WT/WT} and Lmna^{H222P/H222P} mice at 14 weeks (pre-symptomatic; n=6; n=9) and 26 weeks (symptomatic; n=6; n=7) of age. (b) Expression profile of genes encoding enzymes of NAD+ salvage pathway from microarray dataset obtained from 10 weeks old Lmna^{H222P/H222P} mice. (c) Graphs show band intensity of each protein of interest normalized to ERK1/2 from western blot showing expression of Nampt, Nmrk2 and Nmnat1 in lysates of heart from Lmma^{WT/WT} (n=4) and Lmma^{H222P/H222P} (n=4) mice at 26 weeks of age. Western-blot were performed in three independent experiments each performed in triplicates. ERK1/2 was used as a loading control. (d) Graph shows band intensity of Nampt normalized to ERK1/2 from western blot showing expression of Nampt in lysates of human hearts from non-carriers (n=2; controls) and carriers of LMNA mutations (n=2). ERK1/2 was used as a loading control. *p≤0.01; **p≤0.001 and ***p≤0.0001.

FIG. 2: Clinical relevance and potential therapeutic strategy consisting in NR supplementation. (a) Quantification of cellular NAD+ content (pmol/mg tissue) in heart and liver from Lmna^{WT/WT} and Lmna^{H222P/H222P} mice at 26 weeks of age, following 9 weeks of either NR-diet (n=7; n=26) or chow-diet (n=7; n=8). (b) Kaplan-Meier survival curves of Lmna^{WT/WT} and Lmna^{H222P/H222P} following 9 weeks of either NR-diet (n=12; n=15) or chow-diet (n=13; n=11). (c) Box-and-whisker plots showing median fractional shortening (FS) in 17-week- old (pre-treatment), 21-week-old, and 26-week-old (end of treatment) Lmna^{WT/WT} and Lmna^{H222P/H222P} following 9 weeks of either NR-diet or chow-diet (n=12, n=13, n=15, n=11 respectively). Values are shown as 25^{th} to 75^{th} percentiles of the ranked set of data values. The line in the middle is plotted as the median. Whiskers (Tukey method) extend down to the minimum value and up to the maximum value. (d) Expression of Myh7, Nppa and Col1a2 genes by qPCR from cDNAs of heart from Lmna^{WT/WT} and Lmna^{H222P/H222P} mice, at the end of either 9 weeks NR-diet (n=5; n=5) or chow-diet (n=5; n=5). (e) Kaplan-Meier survival curves of Lmna^{WT/WT} and Lmna^{H222P/H222P} following either NR-diet (n=5; n=4) or chow-diet (n=5; n=9). *p≤0.01; **p≤0.001 and ***p≤0.0001

FIG. 3: Activation of the Nmrk2 enzyme in dilated cardiomyopathy. (a, b) RT qPCR analysis of Nmrk2 and Nampt mRNA levels in the hearts of SRF^{HKO} mice at different days (D) after SRF inactivation. (c-e) NAM or NR (30 μmole) were injected i.p. from −D8 to 15 (400 mg/kg/day) to control and SRFHKO mice. Vehicle: saline solution. (c) Myocardial NAD+ levels (Vehicle: N=9 (control), N=8) (SRF^{HKO}). NAM: N=5 (control), N=4) (SRF^{HKO}). NR: N=4 (control), N=4 (SRF^{HKO}). (d, e) RT qPCR analysis of Srf and Nmrk2 mRNA levels. (f, g) Myocardial NAD+ levels in control and SRFHKO mice fed with regular chow diet (CD) or NR enriched diet (0.22% weight/weight) from D5 to 20 (f) or D5 to 50 (g).

Throughout the figure, except for c and f, data are expressed as mean fold change (FC)±SEM over control group. In (f) Asterisks and Hashtag indicate statistical significant difference (T test) between SRF^{HKO} versus control group: *, p≤0.05; **, p≤0.01; ***, p≤0.001 and of NR treatment in the SRF^{HKO} group: #, p≤0.05). In (g), 2-way ANOVA statistical analysis for independent samples showed a genotype effect (¶¶; p≤0.01) and a NR diet effect (§ §; p≤0.01).

FIG. 4: NR supplementation in diet prevents the onset of heart failure and dilatation.

(a) Regular chow diet (CD) or NR supplemented diet (0.22%) was given ad libitum to control and SRFHKO mice from D5 after SRF inactivation to the end of the experiment. Body weight was monitored throughout the period. Data are expressed as mean % weight variation±SEM, compared to weight at D5. ##, p≤0.01 versus groups of the same genotype fed with regular diet. (b-o) Cardiac parameters of control and SRFHKO mutant were analyzed in M-Mode echocardiography between D45 to D47. (b) Heart rate; (c) Left ventricle (LV) mass index; (d) LV ejection fraction; (e) Fractional shortening; (f, g) LV end-systolic diameter (f) and volume (g); (h, i) LV end-diastolic diameter (h) and volume (i); (j, k); LV posterior wall thickness in systole (j) and diastole (k); (I, m) Interventricular septum thickness in systole (I) and diastole (m); (n) LV thickness to radius ratio; (o) Stroke volume. Dimensions were normalized by the body weight. *, p≤0.05 vs control group CD; ##, p≤0.01; ###, p≤0.001 NR versus CD group of the same genotype.

FIG. 5: NR supplementation in diet rescue the loss of citrate synthase activity in the failing heart.

Left ventricle cardiac tissue was isolated at 50 days after tamoxifen injection in control and SRF^{HKO} mice fed with regular chow diet (CD) or NR enriched diet (NR). Proteins were extracted to citrate synthase activity. Data are expressed as mean international units±SEM normalized by the amount cardiac protein quantified by the Bradford assay. **: p≤0.01 versus control CD group. ^{##}: p≤0.01 in comparisons between NR versus CD groups of the same genotype.

FIG. 6: Nmrk2 expression is increased by AMPK and PPARα pathways.

(a) Quantification of total and phospho-protein signal from western blot analyses. Data are normalized on GAPDH signal. The Phos/Total ratio is calculated from GAPDH normalized levels for each individual. N=6 for each group. Data are expressed as mean FC±sem over control group.

(b) NRC were treated with FK866 (10 μM, 72 h), AICAR (500 μM, 48 h) or grown in absence of glucose in the medium (Glc 0) for 48 h and proteins were extracted for western blot analyses. Left: Representative western bot. Right: Quantification on n=3 samples for each condition. Data are expressed as mean FC±sem over NT group.

(c) RT qPCR analysis of Nmrk2 mRNA level in NRC treated as in (D-F).

(d) Nmrk2 promoter deletion analysis by luciferase assay. Rectangle boxes show the position of the putative PPAR binding sites. Data are expressed as in (H, I). *, p≤0.05; *** p≤0.001, ns: non significant, vs the immediately shorter construct.

(e-f) NRC were cotransfected with Nmrk2 luciferase constructs containing 586 or 3009 base pairs of upstream Nmrk2 regulatory region and a dominant negative (DN) AMPK expression vector. NRC were transfected at D3 after plating, followed by AICAR treatment (500 μM) at D4. Luciferase levels were analyzed at D5. Normalized Fireflyl/Renilla values are expressed as fold change of promoterless pGL4 vector.

(g) NRC were co-transfected with the 3009 bp Nmrk2-luciferase construct and the RXR expression vector and with either PPARα, PPAR β/δ or PPARγ expression vectors. NRC were treated 24 h later with the agonists GW7647 [0.6 μM], GW501516 [0.6 μM], and G1929 [0.6 μ4 M], for PPARα, PPARβ/δ and PPARγ, respectively, or with their respective antagonists, GW6471 [10 μM]. GSK3787 [2 μM] or GW9662 [2 μM]. Luciferase levels analyzed at 48 h. Data are expressed as mean FC±SEM over normalized luciferase levels of NRC transfected with p3009-luc construct alone (dashed line) in the same experiment. *, p≤0.05 vs NT cells. ^{##}, p≤0.01 for agonist vs antagonist.

(h) NRC were transfected with the 3009 bp Nmrk2-luciferase construct. Transfected NRC were treated 24 h later with the antagonists G6471, GSK3787, or G9662, 30 minutes before adding AICAR for a further 24 h period. All concentrations as in L. Data are expressed as mean FC±SEM over the p3009-luc construct treated with AICAR alone. *, p≤0.05 vs AICAR treated cells.

(i) Neonate rat cardiac fibroblasts and cardiomyocytes enriched fraction were separated on a discontinuous percoll gradient. Cardiac fibroblasts were transfected with the p-228. p-581 and p-3009-FLuc constructs. Cardiomyocytes were transfected with the p-3009-FLuc. SV40-RLuc construct was cotransfected for normalization. Luciferase activity was analyzed 48 h later. Data are expressed as mean fold change (FC)±SEM over the mean p228-FLuc activity in cardiac fibroblasts.

Student test statistics: *, p≤0.05; **, p≤0.01; ***, p≤0.001 vs reference control group. ^{##}, p≤0.01 between 2 groups as indicated by brackets

FIG. 7: Beneficial impact of nicotinamide riboside treatment on cardiac function and NAD+ levels following transverse aorta constriction (TAC) triggering cardiac hypertrophy and heart failure.

A, Left ventricle ejection fraction (LVEF) and B, Interventricular septum thickness in diastole (IVSThD) was assessed by echocardiography before the surgery (baseline), 2 weeks, 4 weeks and 6 weeks after TAC in sham and TAC groups, with or without NR (400 mg/kg/day) as indicated. (C-H) Mice were sacrificed at 6 weeks after surgery in each group, TAC in SHAM (white bars) and TAC (black bars) groups fed with regulat chow diet (CD) or NR (400 mg/kg/day). C-E, Myocardial NAD+ (C) and NADH (D) levels were quantified by the NAD+ recycling method and calculated NAD+/NADH ratio (E). F-H, RT qPCR analysis of mRNA levels for Nmrk2 (F), Nmrk1 (G) and Nampt (H). Hprt was used as a reference gene. Data are expressed as fold change ratio compared to SHAM-CD group. Throughout the figure, data are expressed a mean+/−s.e.m. Statistics: 2-way Anova for independant factors: §, § §, § § §, significant group effect (TAC versus SHAM) at p<0.05, 0.01, 0.001, respectively. ¶¶¶, significant treatment effect (NR versus CD) at p<0.001. i, interaction effect at p<0.05. Post-hoc Tukey test were performed between groups when interaction was significant: *, **, p<0.05 and p<0.01, respectively.

FIG. 8: Quantification of western blot for NMRK2 and NAMPT proteins in control healthy hearts (n=4) and human failing hearts from patients with non obstructive cardiomyopathy (n=4). Band intensities were quantified using image J software using the background deduction method. The mean ratio of NMRK2 to NAMPT signal in control hearts was set to 1. Data are expressed as mean fold change+/−s.e.m. Statistics: a T-test was applied to the data: **, p<0.01.

### EXAMPLE 1

### Material & Methods

Patients

Left ventricular samples from explanted hearts of two patients carrying LMNA mutation and from autopsied heart of two healthy people were obtained from Myobank-AFM (Paris, France) and National Disease Research Interchange (Philadelphia, Pa., USA) respectively. Patient 1 (P1) carrying the LMNA c.781-783de1AAG, p.Lys261del mutation was a 23-year-old man and patient 2 (P2) carrying the LMiVA c.178C>G, p.Arg60Gly mutation was a 47-year-old woman both developed dilated cardiomyopathy. The control 1 (C1) was a 15-year-old woman died of overdose and the control 2 (C2) was a 57-year-old man died of cerebrovascular accident. All tissue samples were obtained with appropriate approvals and consent (not specifically for this study) from the Institut de Myologie and the National Disease Research Interchange and provided without patient identifiers.

Animals

Mice were bred in an accredited animal facility (accreditation number: C-75-13-08). All experiments on mice were approved by the Comité d'éthique en expérimentation animate Charles Darwin No 5. Mice Lmna^{H222P/H222P} were backcrossed 8 times to 129S2/SvPasOrlRj strain mice to obtain pure genetic background. Male mice used for the studies were maintained under a 12 h light/12 h dark cycle at constant temperature (23° C.) with free access to food and water. Nicotinamide riboside (NR) complementation: Animals at 17 weeks of age were fed for 9 weeks with either a chow diet (A04: Scientific Animal Food & Engineering) or a chow diet complemented with 0.25% of NR (kindly provided by ChromaDex, Inc. California) corresponding to a dose of 400 mg/kg/day for a mouse weighing 25 g eating 4 g of food per day. Nicotinamide (NAM) treatment: Animals at 17 weeks of age were injected intraperitoneally (IP) every two days (q.a.d.) for 9 weeks with either physiologic serum or physiologic serum added with NAM at 50 mg/ml corresponding to a dose of 500 mg/kg for a mouse weighing 25 g.

For survival analysis, Lmna^{H222P/H222P} mice were fed chow supplemented with 0.25% nicotinamide riboside when they were 17 weeks of age and continued until they suffered from significant distress or died. p≤0.001

Statistics

For all animal experiments, the sample size required to achieve adequate power was estimated using the IPSUR module of R software; mean difference=8, SD=10, standard error α=0.05 and power of test β=0.8, type of analysis: t-test; hypothesis: unilateral.

Animals were randomly allocated to individual groups and nonparametric Mann-Whitney U-test was performed to identify significant differences between two groups of interest.

Values for real-time q-PCR, immunoblots and NAD+ content were compared using an unpaired Student t-test. Comparisons of echocardiographic parameters between NR and NAM-treated and placebo-treated LmnaH222P/H222P mice were performed using a Welch t-test; to validate these results, a non-parametric test (Mann-Whitney) was performed and concordance checked. Statistical analyses were performed using GraphPad Prism software.

Echocardiography

Mouse transthoracic echocardiography was performed using an ACUSON 128XP/10 ultrasound with a 11 Mhz linear probe (Mountain View).MHz transducer applied to the chest wall. Mice were maintained under anesthetized with 0.75% isoflurane anaesthesia (0.75% in O2) and placed on a heating pad (25° C.). Left ventricular (LV) parameters were obtained from M-mode recordings in a modified short axis view. The intra ventricular septum (IVS), LV posterior wall (LVPW), LV diameter (LVD) in diastole and systole, the percentage of fractional shortening (FS) and the heart rate (HR) were measured from the mean of at least three separate cardiac cycles. A “blinded” echocardiographer, unaware of the genotype and the treatment, performed the examinations.

NAD+ Concentration Quantification

Fresh mouse tissues were homogenized and processed according to previous report (Sander B J, Oelshlegel F J & Brewer G J (1976) Quantitative analysis of pyridine nucleotides in red blood cells: a single-step extraction procedure. Anal. Biochem. 71: 29-36). The concentration of NAD+ in heart tissue was determined using a spectrophotometric assay at 550 nm based on an alcohol dehydrogenase cycling reaction, in which a Methyl Thiazolyl Tetrazolium (MTT) is reduced to Formazan. Absorption measurements were carried out in a 96 well microplates by spectrophotometry (Tecan, Infinite® M1000 PRO).

Real Time Quantitative PCR

Total RNAs were extracted from around 5 mg of mouse heart using RNeasy Mini kit (Qiagen). RNAs integrity were controlled on the Bioanalyzer 2100 (Agilent). First-strand cDNA was synthesized from total 200 ng of total RNA using the SuperScript III synthesis system (Invitrogen). Real-time PCR was performed with the LightCycler® 480 (Roche) using SYBR Green I Master mix (Roche). PCR was carried out using 40 cycles at an annealing temperature of 60° C. Relative mRNA transcript levels calculated using the 2^{ΔCT} method were normalized by comparison to housekeeping mRNA.

Immunoblotting

Total proteins were extracted in a glass teflon potter from around 5 mg of mouse heart in 200 ml of Cell Lysis Buffer (Cell Signaling) completed with Deacetylation Inhibitors Cocktail (Santa Cruz). Total protein concentration was assessed with BCA™ Protein Assay (Pierce). Total proteins, 15 μg, were separated by SDS-PAGE, transferred to nitrocellulose membranes and blocked in 5% milk or 5% BSA, in 1× TBS-T for 1 h before incubation in primary antibody overnight at 4° C. Primary antibodies used were against Nampt (Abcam: ab45890), Nrnrk2 (Abgent: AP2791c), Nmnat1 (Santa Cruz Biotechnology: sc-30841), Parp-1 (Santa Cruz Biotechnology: sc-7150), AIF (Abcam : ab32516), cleaved caspase 3 (Cell Signaling : #9661), PAR (Calbiochem: AM80) and ERK1/2 (Santa Cruz Biotechnology: sc-30841). Secondary antibodies were horseradish peroxidate-conjugate (Jackson ImmunoResearch). Recognized proteins. were visualized by enhanced chemiluminescence kit ImmobilonTmWestern (Millipore). The signal was captured with a CCD camera (G:Box Syngene) and quantified with ImageJ imaging Software (imagej.net).

### Results

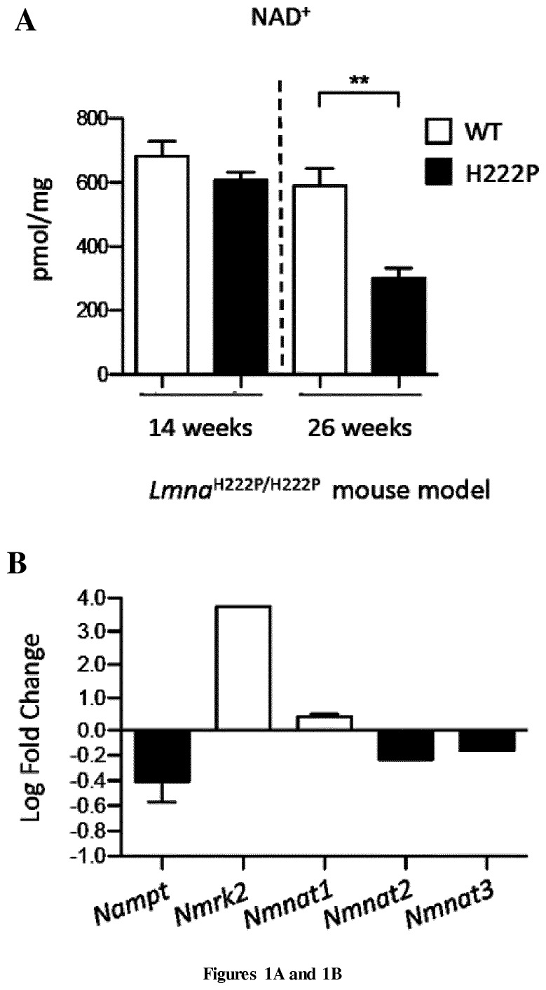

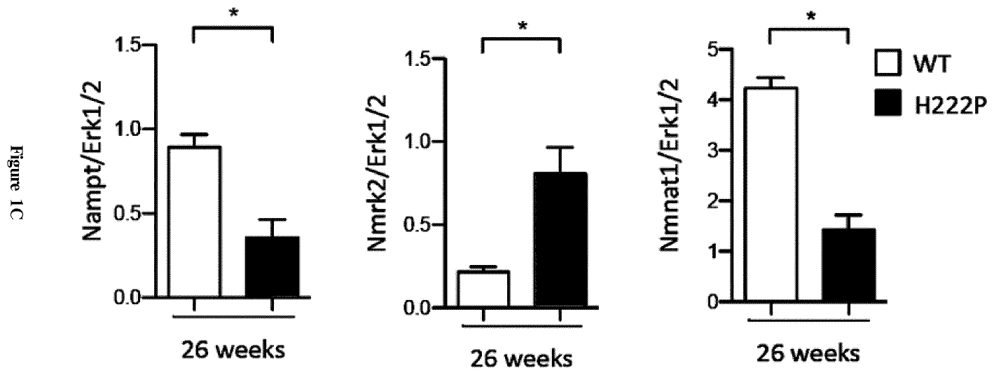

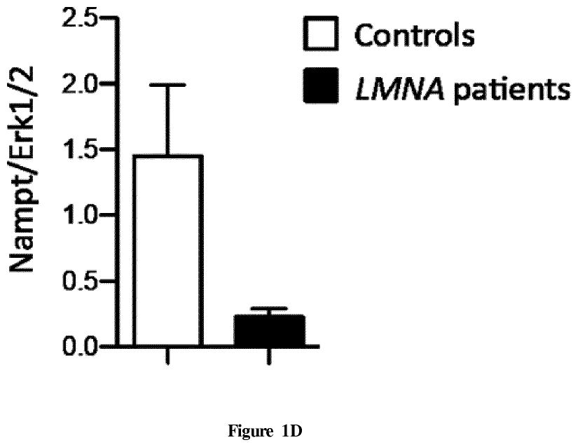

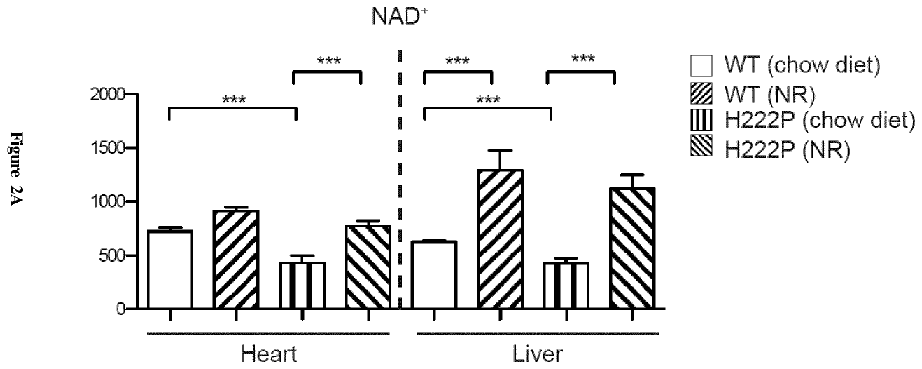

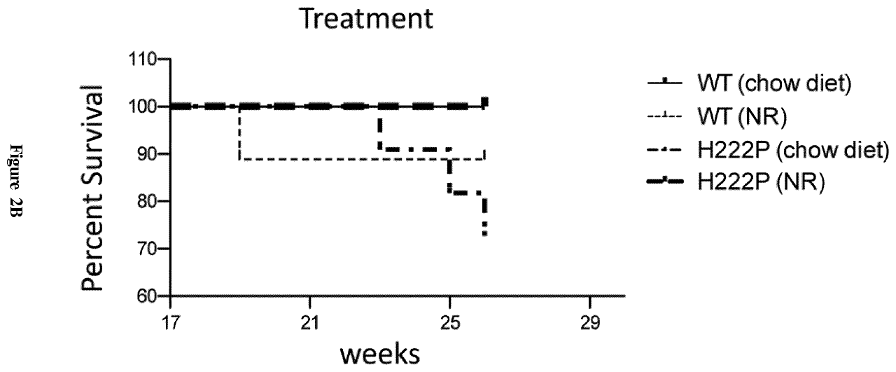

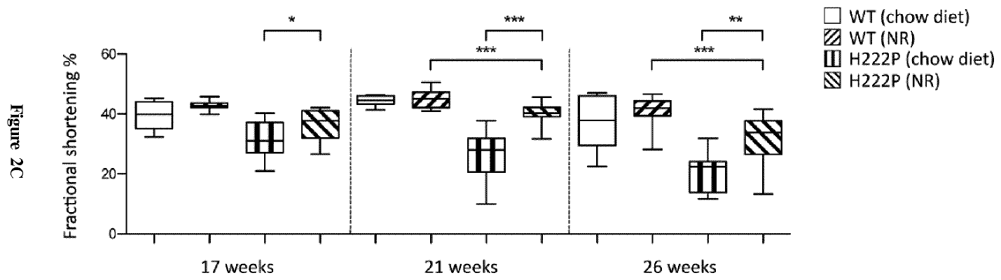

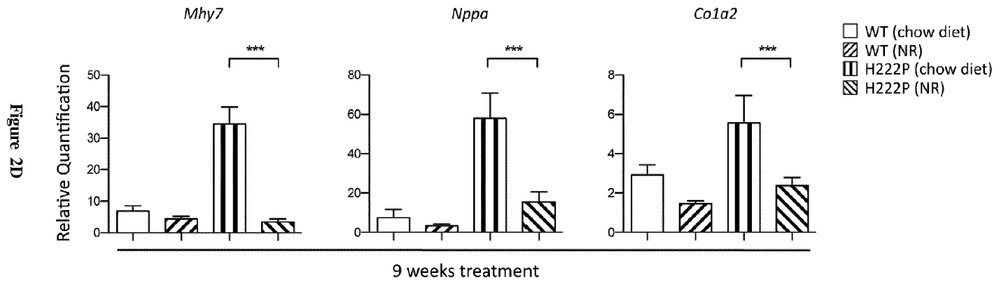

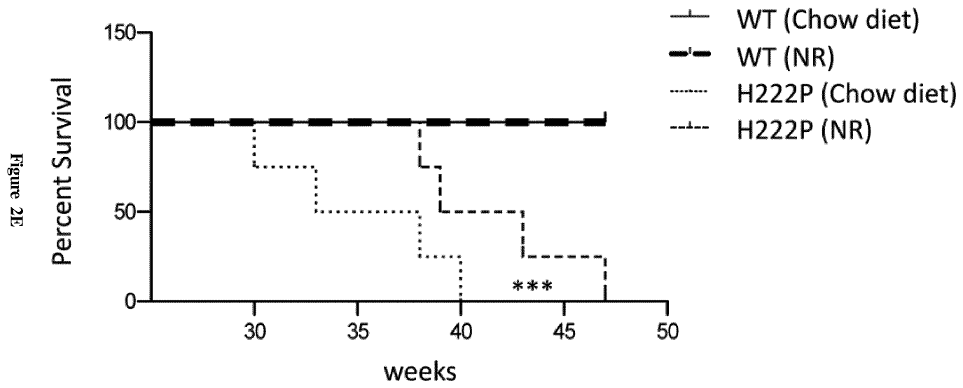

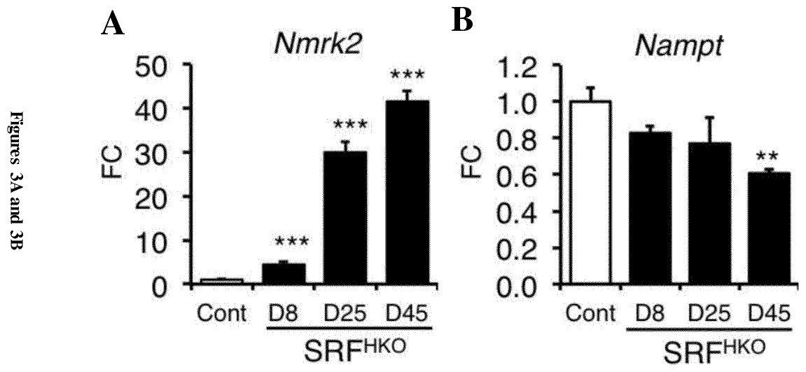

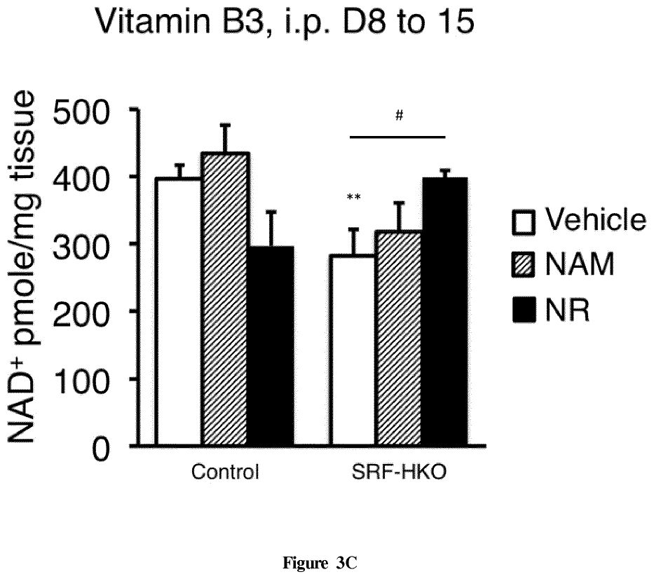

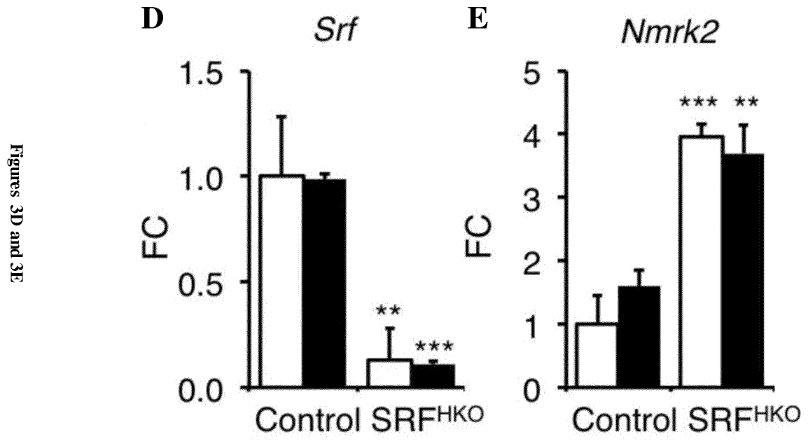

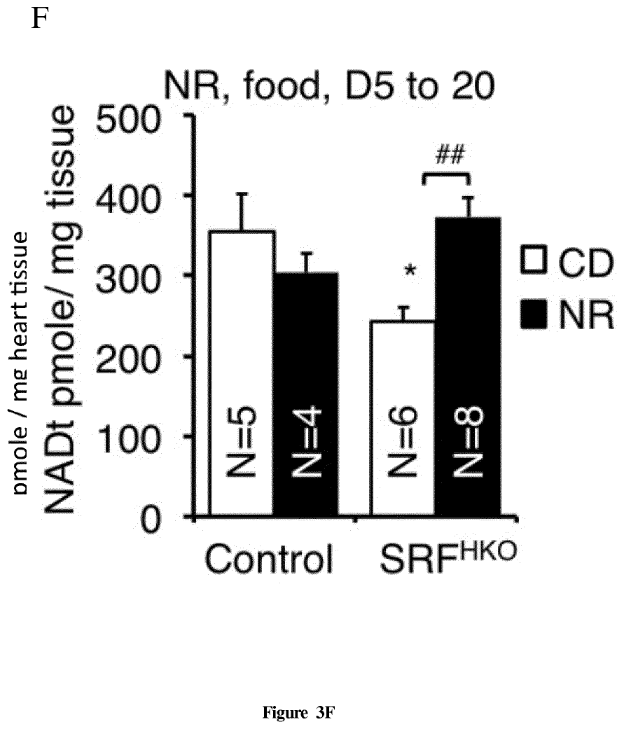

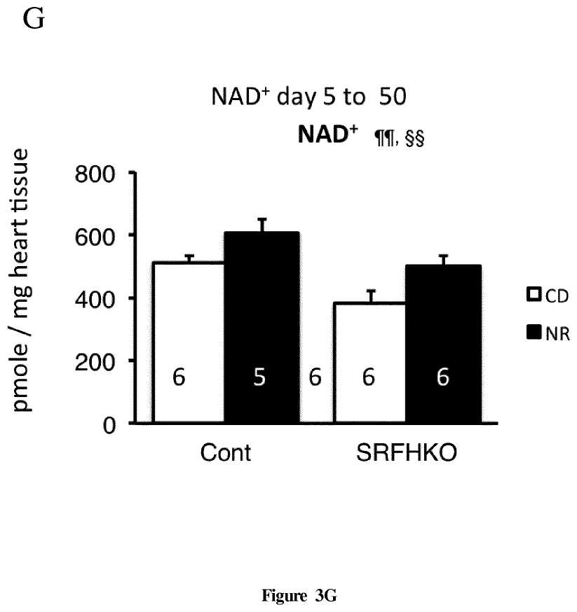

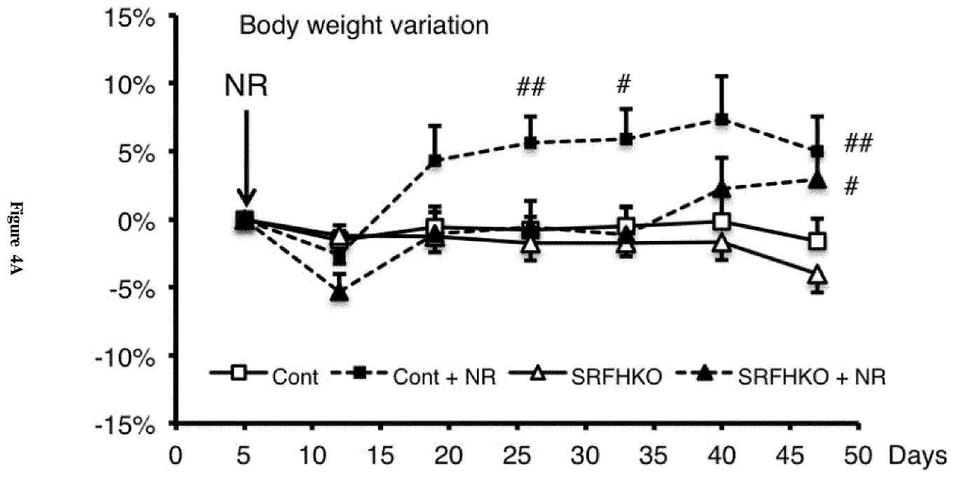

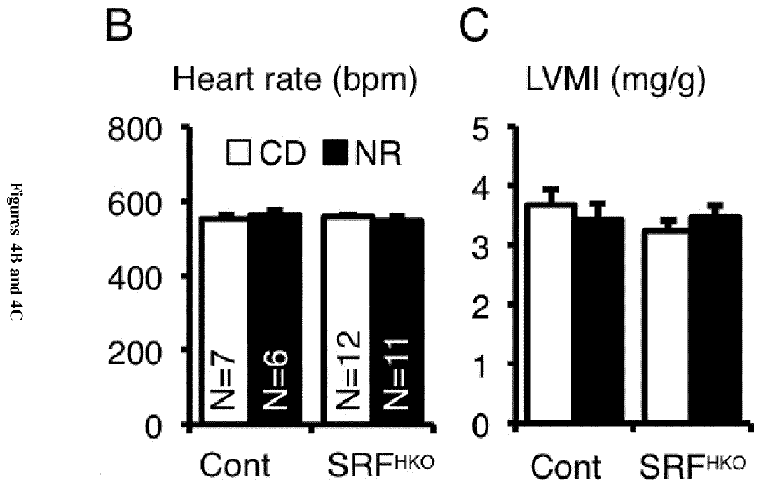

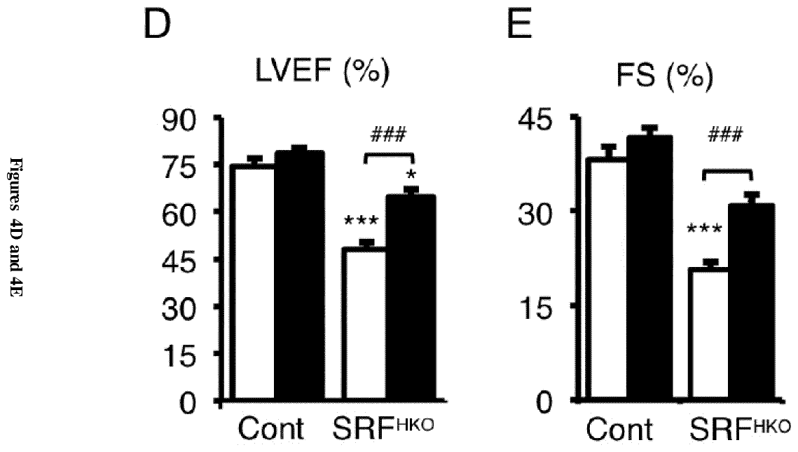

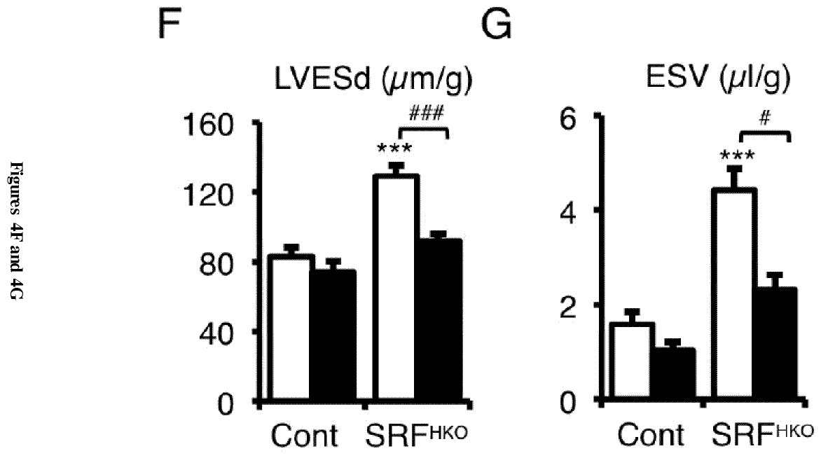

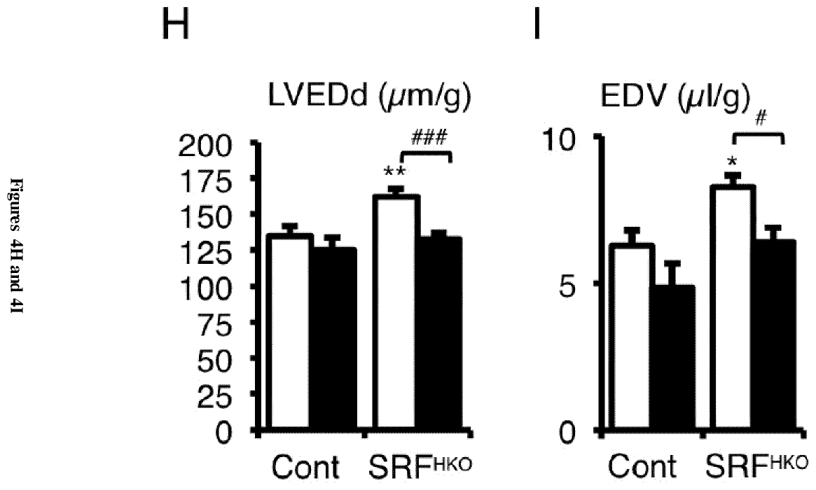

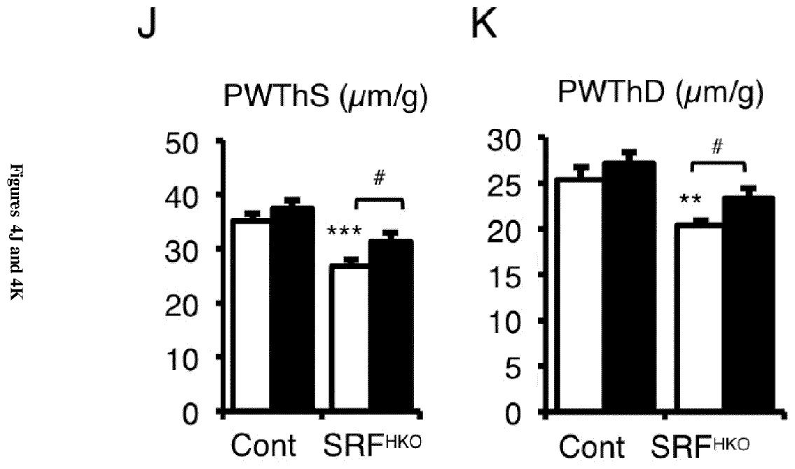

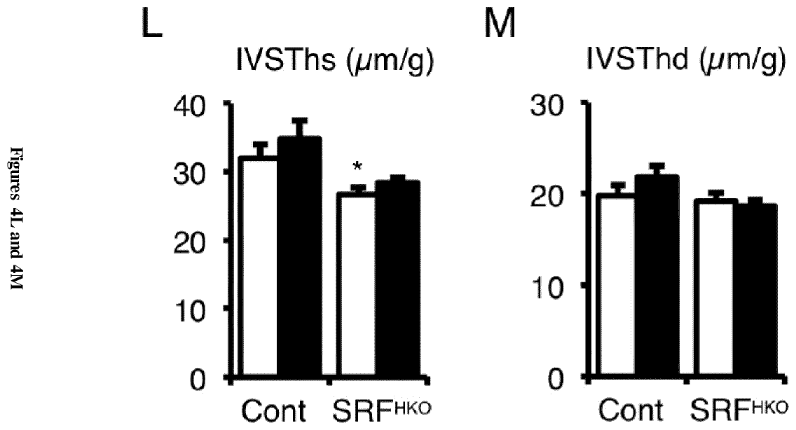

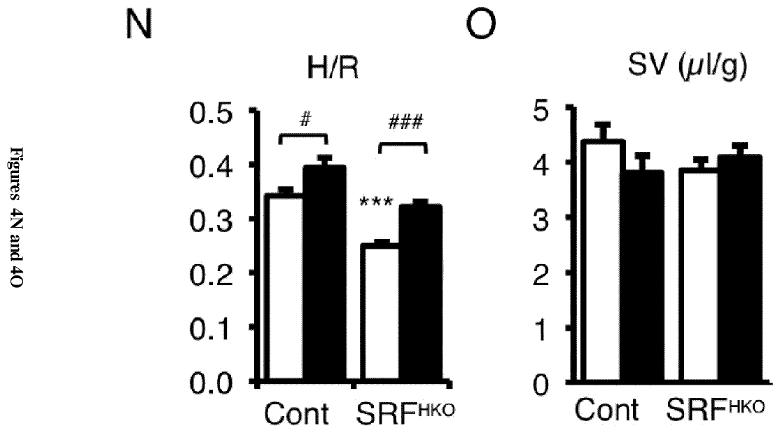

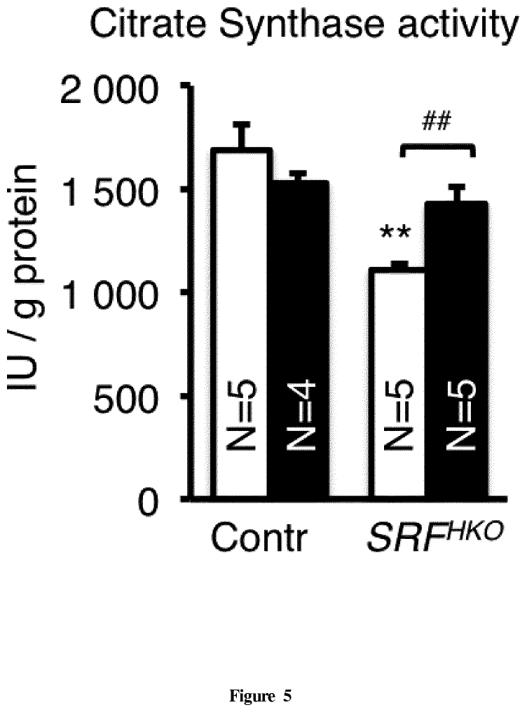

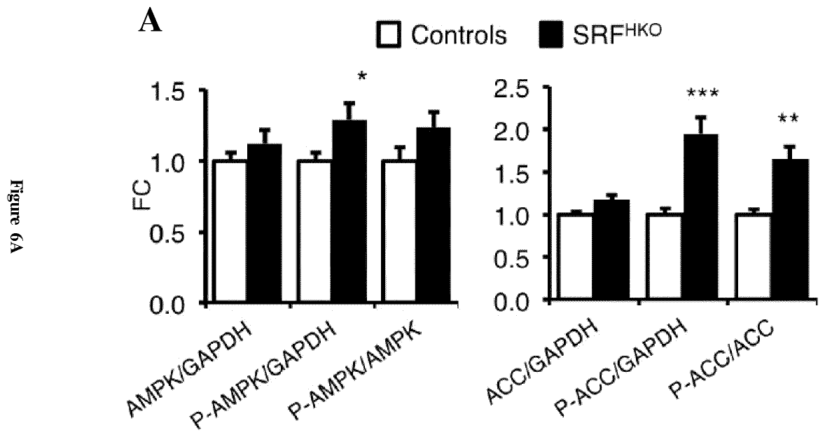

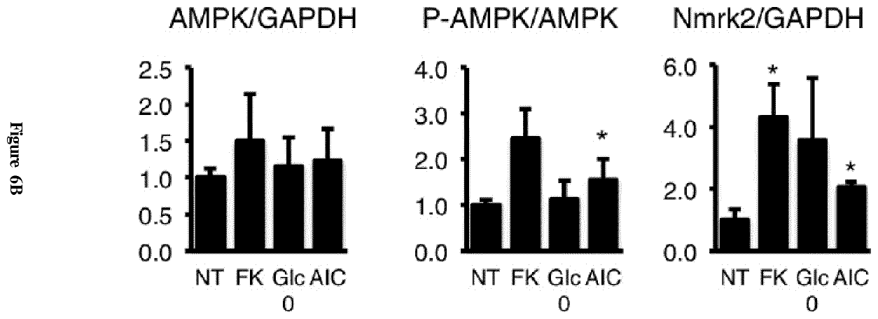

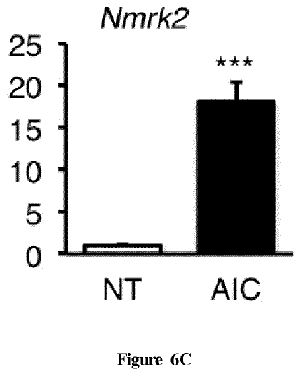

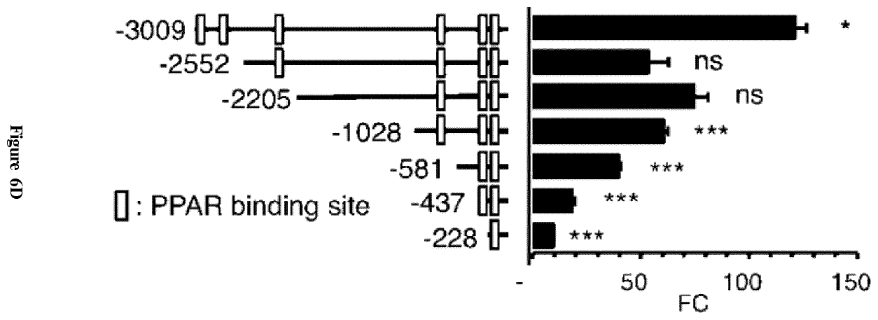

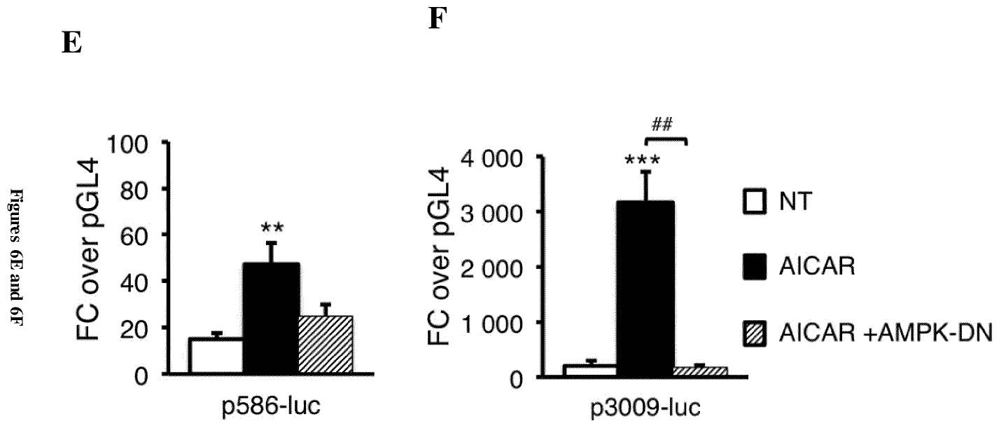

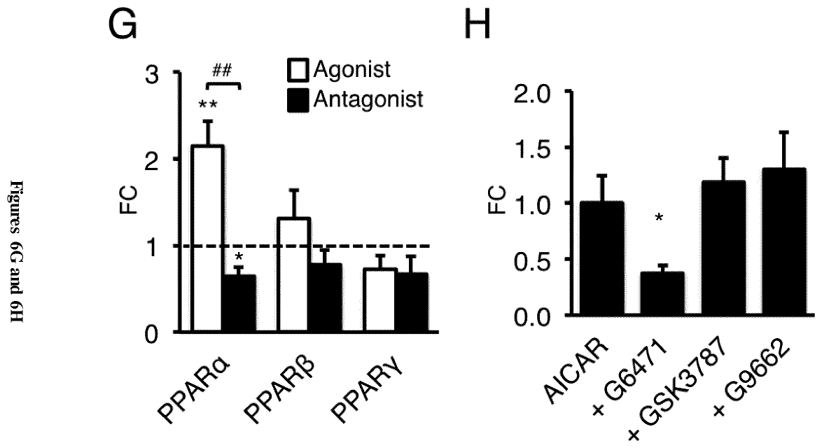

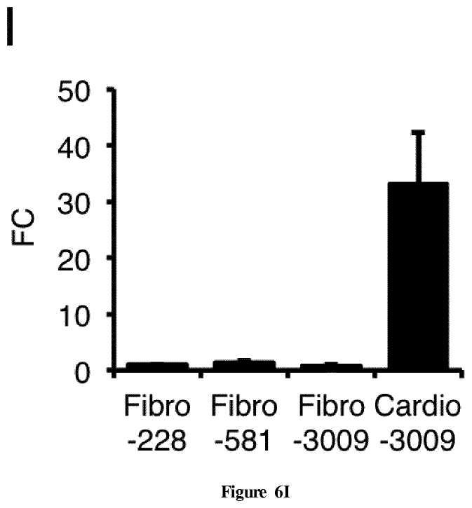

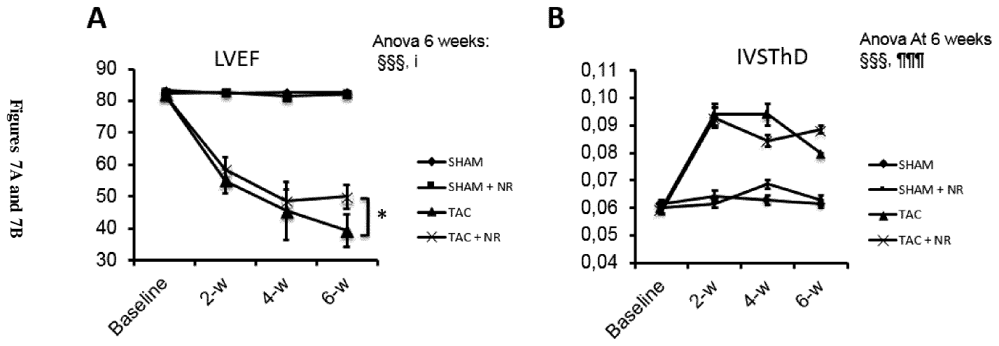

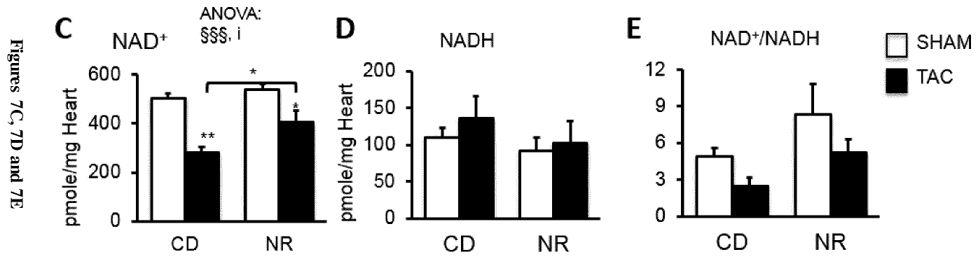

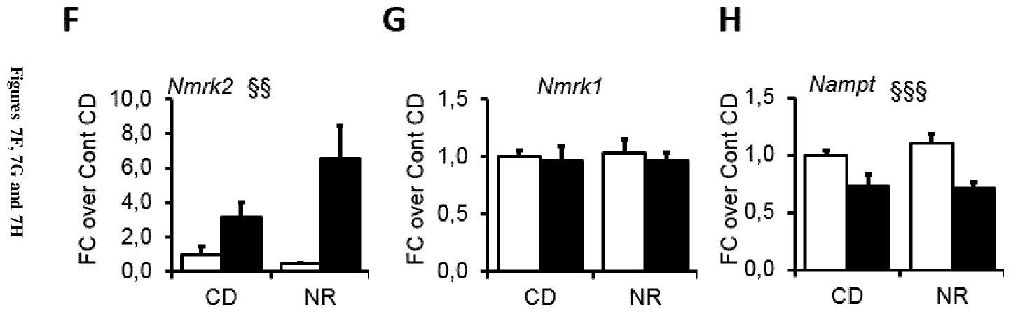

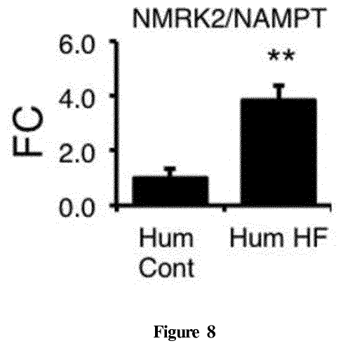

To explore the role of NAD+ salvage pathway in the development of dilated cardiomyopathy, we used a mouse model that display a Lmna mutation changing the histidine in position 222 into a proline, p.H222P. This Lmna p.H222P corresponds to a naturally occurring human disease-causing mutation associated with dilated cardiomyopathy. The homozygous mice Lmna^{H222P/H222P} were shown to have a progressive contractile dysfunction, cardiac remodeling and end stage heart failure to die by 32-34 weeks of age. Given that a consequence of altered NAD+ salvage pathways would lead to an aberrant cellular NAD+ content, we first assessed the cardiac structure and function by echocardiography and the steady-state cardiac NAD+ level in both Lmna^{WT/WT} and Lmna^{H222P/H222P} mice. We showed that NAD+ was significantly lowered in hearts from Lmna^{H222P/H222P} mice when the left ventricular fractional shortening was altered at 26 weeks (symptomatic) (FIG. 1a). There was a correlation between altered cardiac function and cardiac NAD+ content, as there were no decrease NAD+ level in the heart of Lmna^{H222P/H222P} mice at 14 weeks (pre-symptomatic) when the left ventricular fractional shortening was not different from Lmna^{WT/WT} mice (FIG. 1a). We previously examined differential expression of mRNAs isolated from hearts of 10-week old Lmna^{WT/WT} and Lmna^{H222P/H222P} mice using Affymetrix Mouse Genome 430 2.0 Arrays. These data are publicly available at Gene Expression Omnibus with accession numbers GSE6397 and GSE6398. Analysis of these datasets using GEO2R software showed that Nampt expression was lower in the hearts of Lmna^{H222P/H222P} mice compared with controls (FIG. 1b). Cardiac Nmrk2 expression was higher in Lmna^{H222P/H222P} compared with controls (FIG. 1b). The expression of the three Nmnat isoforms (Nmnat1, 2 and 3) was not significantly different from the mutated mice compared with the control animals (FIG. 1b). The same expression pattern of these key genes was also found in two others mouse model of dilated cardiomyopathy (GEO accession number: GSE54838 and GSE17478). We validated the expression level of these selected transcripts by real-time PCR using mRNA extracted from Lmna^{H222P/H222P} and Lmna^{WT/WT} mouse hearts at 14 weeks and 26 weeks. There was a correlation between real-time PCR results and altered expression detected by microarrays for these genes with a significant decreased of Nampt mRNA expression at 14 weeks of age (1.5 fold) and at 26 weeks of age (2.25 fold) and a significant increase of Nmrk2 mRNA expression at 14 weeks of age (4.6 fold) and at 26 weeks of age (11 fold) compared with control mice. In addition, Nmnat1 mRNA expression (the nucleus isoform) was decreased at 14 weeks of age (1.55 fold), and both Nmnat1 and Nmnat3 mRNA expression (the cytosolic isoform) was significantly decreased at 26 weeks of age (2.95 fold and 2.61 fold respectively) compared with control mice. The expression of Nmnat2 (the mitochondria isoform) was not altered in the heart from Lmna^{H222P/H222P} mice. We then evaluated the expression of Nampt, Nmrk2 and Nmnat1 at the protein level, in hearts from Lmna^{WT/WT} and Lmna^{H222P/H222P} mice. The dysregulation of the protein expression level of Nampt, Nmrk2 and Nmnat1 was in concordance with Nampt, Nmrk2 and Nmnat1 mRNAs expression level in hearts from Lmna^{H222P/H222P} mice compared with Lmna^{WT/WT} mice at 26 weeks of age (FIG. 1c). We obtained samples of heart tissue from two human subjects with LMNA cardiomyopathy sampled after cardiac transplantation. Control heart samples were obtained from age matched human subjects with no LMNA mutation. Immunoblotting using antibody against Nampt showed obvious decreased expression in heart tissue of the patients with LMNA mutations compared with controls (FIG. 1d). Taken together these results showed that the metabolism of NAD+ is altered in the heart of Lmna^{H222P/H222P} mice. This aberrant regulation is also observed in patients with LMNA cardiomyopathy.

Given that cardiac Nmrk2 was increased in Lmna^{H222P/H222P} mice, we next tested whether NR, the substrate for Nmrk2, could increase NAD+ content in the heart and therefore blunt the development of left ventricular dysfunction. The animals were fed with NR complemented diet (400 mg/kg/day), starting at 17 weeks of age for 9 weeks. We first determined the NAD+ content of NR-diet Lmna^{H222P/H222P} mice and compared with chow-diet Lmna^{H222P/H222P} mice. We also treated Lmna^{WT/WT} mice and similarly, assessed the NAD+ content. Lmna^{H222P/H222P} mice fed with NR showed an increase of their NAD+ content in both the liver and the heart (FIG. 2a). Feeding Lmna^{WT/WT} mice with the same dosage of NR has the same significant benefit on cellular NAD+ content in both the liver and heart (FIG. 2a). Over the course of the treatment protocol, 38% of chow-diet Lmna^{H222P/H222P} mice died while none of NR-diet Lmna^{H222P/H222P} mice died (FIG. 2b). The overall heart size of NR-diet Lmna^{H222P/H222P} mice was reduced compared with chow-diet Lmna^{H222P/H222P} mice. Cardiac structure and function were assessed by echocardiography after 4 and 9 weeks of NR-treatment in the four different groups studied. No difference was observed across the wild type mice whatever the diet and the course of the treatment (FIG. 2c). Compared with chow-diet Lmna^{H222P/H222P} mice, NR-diet treated Lmna^{H222P/H222P} mice had significantly decreased left-ventricular end systolic (LVDs) and left-ventricular end diastolic (LVDd) diameters. They also have improved cardiac contractility indicated by increased left-ventricular fractional shortening (FS) starting as early as four weeks of treatment (FIG. 2c). Hence treatment with NR for 9 weeks delayed the development of left-ventricular dilatation and cardiac contractile dysfunction in Lmna^{H222P/H222P} compared with Lmna^{WT/WT} mice, Lmna^{H222P/H222P} mice had significantly increased expression of Mvh7, Nppa and Col1a2, which encode the β-myosin heavy chain, the natriuretic peptide type A and the collagen, type 1, alpha 2 (FIG. 2d). Treating Lmna^{H222P/H222P} mice with NR leads to a significant lowering of these markers of cardiac remodeling compared with chow-diet treated animals (FIG. 2d). The percentage of survival of NR-diet Lmna^{H222P/H222P} was accessed in a pilot study, starting NR feeding at 17 weeks of age. Chow-diet treated Lmna^{H222P/H222P} mice started to die at 30 weeks with a mean of survival at 33 weeks while NR-diet treated Lmna^{H222P/H222P} started at 38 weeks with a mean of survival at 39 weeks (FIG. 2e). Taken together these results showed that a treatment with NR resulting in an increase of cardiac NAD+ is efficient to partially restore the left ventricular function in a mouse model of LMNA cardiomyopathy and increase the lifespan of the mice.

As Nampt expression level is decreased in Lmna^{H222P/H222P} mice (FIG. 1c), this suggests that NAD+ salvage through NAM, the substrate of Nampt, might not be efficient in these mice. Thus, treating Lmna^{H222P/H222P} mice with NAM should neither restore their cardiac NAD+ content nor improve their left ventricular dysfunction. We treated Lmna^{H222P/H222P} mice with NAM (500 mg/kg/q.a.d.) starting at 17 weeks of age, for 9 weeks. Lmna^{H222P/H222P} mice injected with NAM did not exhibit any changes of their cardiac NAD+ content in both the liver and the heart compared with placebo animals. Left ventricular structure and function were assessed by echocardiography after four weeks of treatment and at the end of the NAM-treatment. Compared with placebo Lmna^{H222P/H222P} mice, NAM-treated Lmna^{H222P/H222P} mice had no significant changes in left-ventricular end systolic, left-ventricular end diastolic diameters, and cardiac contractility. NAM-treated Lmna^{H222P/H222P} mice had no changed expression of Myh7, Nppa and Col1a2. Taken together these results demonstrated that treating Lmna^{H222P/H222P} mice with NAM is not efficient to restore the cardiac NAD+ content and the cardiac left ventricular function and that the decreased Nampt expression level is one of players of the cardiac phenotype.

We then asked how the decreased of cardiac NAD+ content could trigger the contractile dysfunction in LMNA cardiomyopathy. NAD+ could be consumed by members of the poly(ADP-ribose) polymerase (PARPs) family. The level of PARylation detected by poly(ADP-ribose) (PAR) monoclonal antibody is significantly decreased in the hearts from Lmna^{H222P/H222P} mice compared with control animals. Given that PARP enzymes bind and cleave NAD+ to produce NAM and couple one or more ADP-ribose units to promote PARylation of acceptor proteins, we assessed the level of cardiac PARP-1, the most abundant isoform of the PARP enzyme family. We showed that the protein PARP-1 cardiac content was also lowered in the mutated mice. PARP-1 has been described as playing a major role for efficient DNA repair upon DNA damage, which has biological implication for cell death. We showed that the expression of both the apoptosis-inducing factor (AIF) and cleaved-caspase 3 was decreased in the Lmna^{H222P/H222P} mice compared with Lmna^{WT/WT} mice, suggesting a role played by PARP-1 in cell death program in LMNA-cardiomyopathy. Treating Lmna^{H222P/H222P} mice with NR leads to an increase of PARylation as well as an activation of PARP-1. AIF and cleaved-caspase 3.

### Discussion

Altered NAD+ metabolism through the salvage pathway has been described in muscular dystrophies and metabolic alteration. To the best of our knowledge, this is the first time that altered NAD+ salvage has been reported in heart failure and cardiomyopathy. We demonstrate here that oral supplementation with NR, a vitamin B3 form and NAD+ precursor, efficiently prevented development and progression of LANA cardiomyopathy in mice. We show that NR significantly delayed disease progression even in mice that already manifested the disease, which is the time frame when medicated treatment would typically be started for patients. These results are remarkable, as they underline the utmost importance of specific vitamin co-factors, as modifiers of metabolism in cardiac disease, and emphasize the role of nutritional signaling in the pathogenesis of LMNA cardiomyopathy. The results presented in this study are in line with previous work showing that exogenous NAD+ content blocks cardiac hypertrophy. A recent study demonstrated that NR treatment transiently rescued the cardiomyopathy in iron deficiency mouse model. Similarly, increasing the NAD+ content using precursors of NAD+ has been proven efficient to rescue hallmark of mitochondrial myopathy both in mouse and human. Given that vitamin B3 supplement is potentially translatable into therapy, our work supports more studies on human to begin to evaluate the therapeutic benefits of increasing NAD+ and assess the benefit of such therapy on cardiomyopathy.

How NAD+ results in cardiac tissue injury in LMNA cardiomyopathy is yet to be fully elucidated, but inference can be made from related experiments. PARP enzymes bind and cleave NAD+ to nicotinamide and negatively charged poly(ADP-ribose) (PAR) polymers on a large set of target proteins. Given its DNA binding zinc fingers, PARP can be activated through DNA strand breaks and irregular DNA structures. PARP-1 has been extensively studied and implicated in several cellular processes as diverse as cell cycle regulation, differentiation, DNA repair, inflammation, metabolic regulation, RNA processing and transcription. In the attempt to repair DNA lesions, PARP activation takes place to facilitate the DNA repair machinery. The inhibition of PARP-1 by the decreased cardiac NAD+ content in Lmna^{H222P/H222P} mice could lead to either cell death, or to genomic instability, which could participate to the pathogenesis of LAMA cardiomyopathy. The partial loss of PARP-1 activation in Lmna mice could have consequence on DNA damage repair and apoptosis.

In conclusion, our experiments demonstrate a novel contributory metabolic mechanism of dilated cardiomyopathy triggered by altered NAD+ salvage pathway. Moreover, we have also shown fine tuning of cellular NAD+ content that the metabolic target, Nmrk2, is susceptible to therapeutic use by nicotinamide riboside feeding, is a straightforward therapeutic strategy that should be explored in patients with LMNA cardiomyopathy.

### EXAMPLE 2

### Material & Methods

Patients

Left ventricular myocardium was obtained from terminally failing human hearts of 4 patients (mean age 54 years±7, S.D.) at the time of transplantation at the “Hôpitaux Universitaires de Strasbourg” (HUS) as previously published with approval of HUS ethics committee Garnier A, Zoll J, Fortin D, N'Guessan B, Lefebvre F, Geny B, Mettauer B, Veksler V, Ventura-Clapier R. Control by circulating factors of mitochondrial function and transcription cascade in heart failure: A role for endothelin-1 and angiotensin ii. Circ Heart Fail. 2009; 2:342-350). Patients' characteristics are detailed in Table S2. Human cardiac tissue control samples (LV, mean age 51 years±4.5, S.D) were obtained from general organ donors whose non-diseased hearts were explanted to obtain pulmonary and aortic valves. The investigations conformed to the principles of the Declaration of Helsinki. Experimental protocols and were approved by the Ethical Review Board of the Medical Center of the University of Szeged and by the Scientific and Research Ethical Committee of the Medical Scientific Board at the Hungarian Ministry of Health (ETT-TUKEB; No. 51-57/1997 OEj and 4991-0/2010-1018EKU).

Transgenic mice. All experiments with animals conformed to the Directive 2010/63/EU of the European Parliament and were approved by the ethics committee Charles Darvin #5 (agreement 00369.01). SRF^{HKO} mice bear the tamoxifen-inducible α-MHC-MerCrMer transgene and the Siffloxed allele (Sf/Sf) as described previously (Parlakian et al. (2005). Temporally controlled onset of dilated cardiomyopathy through disruption of the SRF gene in adult heart. Journal 112, 2930-2939; Tritsch et al. (2013). An SRF/miR-1 axis regulates NCX1 and Annexin A5 protein levels in the normal and failing heart. Journal 98, 372-380). Transgenic mice have been bred for more than 20 generations in C57BL/6N background. The mice were housed in standard animal facility (CEF. Hôpital Pitié Salpétrière, agreement A-75-13-15, 75013 Paris), under a 12-h dark/light cycle. Tamoxifen (Sigma T5648) was administrated by i.p. injection at a dose of 0.7 mg diluted in 100 μl peanut oil at day (D) 0, D1 and D2. (Sf/Sf) littermates injected with tamoxifen at the same time than mutant were used as controls.

Phenylephrine Administration.

The series of mice treated with PE and the impact of the treatment on cardiac parameters have been published previously (Tritsch et al. (2013). An SRF/miR-1 axis regulates NCX1 and Annexin A5 protein levels in the normal and failing heart. Journal 98, 372-380). Briefly. PE was administrated at 80 mg/kg/day for 15 days with osmotic micropumps (Alzet) implanted in the back skin of adult mice under anesthesia (2% Isoflurane. 2% oxygen).

Transverse Aorta Constriction (TAC)

Transverse aorta constriction was performed in 2 month-old male mice under anesthesia (ketamine 100 mg/kg, xylazin 16 mg/kg). Skin was opened above the sternum and the upper part of the sternum was cut on a length of 2 mm allowing the mouse to breath without the need of a ventilator. A surgical suture was passed under the aortic cross between the right brachiocephalic (innominate) artery and left common carotid artery and a 27-½ gauge blunt needle was placed parallel to the wall of the aortic cross as a calibrator. Two loose knots were tied around the aortic cross and the needle. The first knot was quickly tied against the needle, followed by the second and the needle was promptly removed in order to produce a constriction of 0.4 mm in diameter. In sham, mice the procedure was the same except for the constriction of the aorta. NR treatment was started 2 days after the TAC or SHAM surgery.

Nicotinamide and nicotinamide riboside i.p. administration. Nicotinamide was diluted in saline at 300 mM. Nicotinamide riboside (NR) bromide salt stock solution (1M) was in 10 mM citric acid pH 6.0. For injection 3 volumes of NR stock solution was mixed extemporaneously with 7 volumes of PBS to obtain a 300 mM injectable solution at pH 7.2. Mice were injected daily at a dose of 1 μmole/g body weight.

Nicotinamide Riboside Supplementation in Diet

SRF^{HKO} and control (Sf/Sf) mice were administered either rodent maintenance SAFE A04 diet containing 3.10% crude fat. 16.10% crude proteins. 26% starch and 1.9% sugar (Scientific Animal Food Engineering, Paris, France) or SAFE # A04 diet supplemented with NR. NR was provided by Chromadex under brand name NIAGEN (Irvine Calif. USA). The soft pellets were manually prepared every 5 days by mixing 1.65 g of NR into 500 g of powdered SAFE A04 diet and 235 ml of water to reach 2.24 mg of NR/g (wet weight). Control diet was prepared in the same way omitting NR. Mice had ad libitum access to food and water. Mice (average body weight 31.3±0.82 g, no difference between groups) consumed an average of 6 g of soft food per day, reaching a daily intake of 450 mg NR/kg. No difference in food intake was observed between SRF^{HKO} and control mice.

RT-qPCR

cDNAs were reverse transcribed from RNA (1.5 μg) extracted in TRIzol (Thermofisher) from cell cultures and tissues using the Superscript II Reverse Transcriptase (Life Technologies). Quantitative PCR was carried out on a Light Cycler 480 (Roche Diagnostics) using Fast Start SYBR Green Master (Roche Diagnostics). Quantification of gene expression was calculated as R=2^{ΔCt(Ref Cp-target Cp)}, with Hprt used as a as reference. Primers were designed using the NCBI Primer-BLAST software. Primer sequences are available on request.

Western Blot Analysis

Proteins were homogenized in a lysis buffer (Tris-HCl pH 7.5 50 mM, NP40 Igepal 1%, NaCl 150 mM, EDTA 1 mM, DTT 1 mM, Glycerol 10%) in the presence of proteases.

phosphatases and deacetylases inhibitors (PMSF 0.5 mM, NaF 50 mM, PPiNa 5 mM, Roche protease cocktail inhibitor 1/100, Santa Cruz deacetylase cocktail inhibitor 1/100). Equal amounts of proteins (10 to 20 μg) were separated on SDS-PAGE and transferred to nitrocellulose membranes. Proteins were detected by overnight incubation at 4° C. with primary antibodies, followed by IRDye 700 or IRDye800 fluorescent antibodies (Li-Cor Biosciences. 1/2500) and scanned on an Odyssey CLx Infrared Imaging System (Li-Cor Biosciences).

Antibody list

| Target protein | Antibody Supplier. ref # | Dilution |
|---|---|---|
| Acetyl-CoA | Thermoscientific. # MA5-15025 | 1/1000 |
| Carboxylase (ACC) | | |
| ACC Phospho- | Thermoscientific. # PA5-17725 | 1/1000 |
| Ser79 | | |
| AMPKα | Cell Signaling #2532 | 1/1000 |
| AMPKα (Phospho- | Cell Signaling #2535 | 1/1000 |
| Thr172) | | |
| FKHR (H- | Santa Cruz BT.. # sc-11350 | 1/1000 |
| 128)(FoxO1) | | |
| Ac-FKHR (D-19): | Santa Cruz BT..# sc-49437 | 1/200 |
| GAPDH | Sigma #G9545 | 1/3000 |
| Acetyl-Lysine | Cell Signaling #9441 | 1/1000 |
| HA epitope | Sigma #H6908 | 1/500 |
| Nmrk2 (MIBP) | MBL International Corp.. # | 1/200 |
| | K0099-3, mouse monoclonal 5B4.7 | |
| PARP | Cell Signaling #9532 | 1/1000 |
| PAR (Anti-Poly | Pharmingen #551813 | 1/500 |
| (ADP-Ribose) | | |
| p53 (acetyl K386) | Abcam #ab52172 | 1/500 |

Immunofluorescent Staining

Hearts were harvested after cervical dislocation and washed in cold PBS. Frozen hearts section (10 μm) were fixed in 3.7% formaldehyde in PBS at room temperature (RT) for 10 minutes followed by permablization in PBS/Triton X100 (0.2%) for 10 minutes. Endogenous mouse immunoglobulins were blocked by 1 h. RT. incubation with monovalent anti-mouse IgG Fab fragments (Jackson Immunoresearch) followed by saturation of non-specific binding sites with 2% BAS and 10% goat serum. Sections were incubated o/n at 4° C. in anti-MIBP (Nmrk2) (MBL. 1/100) diluted in saturation solution. The day after, sections were incubated with FITC-coupled anti-hVIN-1 vinculin antibody (Sigma. 1/100) and Cy3-couple anti-mouse IgG1 (Jackson Immunoresearch. 1/400). Confocal images were acquired on a Leica SPS microscope with identical gain and offset parameters for all samples.

Echocardiography

Echocardiography was performed on lightly anesthetized mice given isoflurane (induction with 2% isoflurane 100% O2. and maintained with 0.5% isoflurance 100% O2). Non-invasive measurements of left ventricular dimensions were evaluated using Doppler echocardiography (Vivid 7 Dimension/Vivid7 PRO; GE Medical Systems) with a probe ultrasound frequency range of 9-14 MHz. The two-dimensionally guided time-motion recording mode (parasternal long-axis view) of the left ventricle (LV) provided the following measurements: diastolic and systolic septal (IVS) and posterior wall thicknesses (LVPW); internal end-diastolic (LVEDD) and end-systolic diameters (LVESD); and heart rate. Each set of measurements was obtained from the same cardiac cycle. At least 3 sets of measurements were obtained from 3 different cardiac cycles. LV fractional shortening (FS) was calculated using the formula: (LVEDD−LVESD)/LVEDD×100. LV myocardial volume (LVV), LV end-diastolic volume (EDV), and end-systolic volume (ESV) were calculated using a half-ellipsoid model of the LV. From these volumes, LV ejection fraction (EF) was calculated using, the formula: (EDV−ESV)/EDV×100. H/R ratio was calculated by the formula (PWThd+IVSThd)/LVEDd.

NAD Extraction

Tissue frozen powder was resuspended in 75% ethanol, 25% HEPES 10 mM pH 7.1, buffer (20 μl/mg of tissue). Extracts were warmed 5 min at 80° C., then directly cold on ice and centrifuge 5 min at 16 000 g. Tissues extracts were normalized on the weight of tissue used for extraction. NAD was extremely stable in this buffer and resistant to heat degradation over 60 minutes. Using spiking concentrations of known amounts of NAD⁺ and NADH (Sigma-Aldrich) in pure buffered ethanol solution followed by dilutions in acid (HCl 0.1M) or basic (NaOH 0.1M) buffer, heating at 60° C. for 30 minutes and neutralization with TRIS buffer pH 7.1, we found that NAD⁺ was extremely stable after heating in acid condition (>99% recovery) while NADH was completely destroyed (<1% recovery). Heating in NaOH buffer led to some destruction of both NAD⁺ and NADH and this protocol was discontinued. Hence, we selected the protocol of selective acid-heating destruction of NADH in the cardiac samples for the determination of free NAD⁺ versus free NADH concentrations in these extracts, the buffered ethanol extracts were diluted 1/10 in HCl 0.IM and heated 30 minutes at 60° C. to destroy NADH and then neutralized with Tris buffer at pH 7.1 before measuring NAD⁺. After adjustment of dilution factors, NADH levels were calculated by substracting the NAD⁺ concentration to the non-heated ethanolic extract values: NADH=NADt (non heated)−NAD⁺ (heated).

For cell cultures, the wells were washed twice with PBS and scraped on ice in HClO₄ 0.5M (100 μl/well in 12-well plate). Acid extracts were neutralized with a half volume of KOH 1M/K2HPO4-KH2PO4 0.33 M pH 7.5. Heat denaturation was selective for NAD⁺ in this condition. Measures were made on a volume of 12.5 ₁.1.1_, of non heated extracts (NADt) or 25 μl of heated extracts (NADH) (diluted 1/10 to 1/20).

For both types of extracts (buffered ethanol or HClO₄) we added 100 μL of reaction buffer (600 mM ethanol, 0.5 mM 3-(4.5-dimethylthiazol-2-yl)-2.5-diphenyltetrazolium bromide (MTT), 2 mM phenazine ethosulfate (PES), 120 mM Bicine (pH 7.8), yeast alcohol dehydrogenase (SIGMA A3263>300 u/mg.) 0.05 mg/ml. Kinetics of the reaction (OD at 550 nm, every 30 seconds for 40 minutes) was followed on a TECAN Infinite F500 microplate reader. NAD was quantified in duplicates for each sample by comparison to a range of standard NAD⁺ concentration using linear regression curve equation method between NAD⁺ standard concentrations and the slope of the reaction in OD units/sec. NAD⁺/NADH was obtained by the formula ([NADt]−[NADH])/[NADH]. Cell extracts were quantified for protein concentration determined by Bradford assay.

Isolation and Treatments of Neonatal Rat Cardiomyocytes (NRC) NRC were isolated as described previously (Diguet et al., 2011). 1-day-old rat pups were killed by decapitation and the cardiac ventricles were harvested and minced into 1 to 2-mm wide cubes with scissors. After washing with Tyrode solution, heart fragments were subjected up to 10 rounds of digestion with 0.05 mg/ml of Liberase Blendzyme 4 (Roche Applied Science) in 10 ml oxygenated Tyrode solution under agitation at 37° C. for 10 minutes. The supernatant of the first digestion was discarded and the following digestions were centrifuged and the cell pellet dissociated in 2 ml of DMEM, 10% FCS. The different fractions were pooled and centrifuged on a discontinuous Percoll gradient (bottom 58.5%. top 40.5%. 30 min. 3000 rpm) to enrich in cardiomyocytes at the interface between the 2 Percoll solutions. Non cardiac cells containing mainly fibroblasts were at the top of the tubes. Neonatal cardiomyocytes were seeded at a density 5×10⁵ cells/well in 6-well plates coated with 10 μg/ml of laminin (BD Biosciences) in DMEM without pyruvate, with glucose 4.5 g/l, 10% horse serum, 5% FCS and cultured at 37° C. in 1% CO, atmosphere. To assay the impact on NAD⁺ and Nmrk2 expression, Azaserin and FK866 drugs were added in the culture medium at day 5 and renewed everyday when the treatment was prolonged over several days. The same was done for NAD⁺, NR and AICAR (5-aminoimidazole-4-carboxamide-1-β-D-ribofuranoside). AICAR was administrated for 24 h only. Doses are indicated in the legends. For treatment of transfected cells (see next section), AICAR, FK866 and PPAR agonists and antagonist were administrated 1 day after transfection. PPAR agonists: GW7647 for PPARα, GW501516 for PPARβ/δ and GW1929 for PPARγ; PPAR antagonists: GW6471 PPARα, GSK3787 for PPARβ/δ and GW9662 for PPARγ were ordered from SIGMA ALDRICH CHIMIE, Saint Quentin Fallavier, France.

Cloning of Mouse Nmrk2 Promoter in Luciferase Reporter Plasmid and Transfection Procedure

The transcription initiation site of mouse Nmrk2 promoter was determined by 5′RACE and was conform to the reported site in the Genebank (NM_027120.2). The promoter of the mouse Nmrk2 gene was cloned from C57B16/N genomic DNA by PCR, and inserted into a pGL4.10[luc2] luciferase reporter plasmid (Promega prod no E6651). Subsequent deletions were performed by enzymatic digestion or primer mediated subcloning of the original fragment. 2.5×10⁵ cardiomyocytes in 12-well plates were co-transfected at day 2 of culture with 1 μg of Nmrk2 pGL4.10[luc2] constructs and 100 ng of pGL4.73[hRluc/SV40] Vector (Promega prod no E6911) as an internal control of transfection efficiency using Lipofectamine 2000 (Lifetechnology) as indicated by the supplier. In the case of AICAR treatment, Luciferase activity was analyzed 48 h later with the Dual-Luciferase Assay System (Promega).

AMPK, PPAR, RXR Vectors

The pcDNA3 vectors harboring dominant negative AMPK, PPARα, PPARβ and PPARγ as well as RXR genes were kindly provided by Pr. Michel Raymondjean (El Hadri et al. (2015). AMPK Signaling Involvement for the Repression of the IL-1beta-Induced Group IIA Secretory Phospholipase A2 Expression in VSMCs. Journal 10, e0132498; Ravaux et al. (2007). Inhibition of interleukin-1 beta-induced group 11A secretory phospholipase A2 expression by peroxisome proliferator-activated receptors (PPARs) in rat vascular smooth muscle cells: cooperation between PPARbeta and the proto-oncogene BCL-6. Journal 27, 8374-8387; Woods et al. (2000). Characterization of the role of AMP-activated protein kinase in the regulation of glucose-activated gene expression using constitutively active and dominant negative forms of the kinase. Journal 20, 6704-6711). These vectors were cotransfected at a dose of 100 ng/well in 24-well plates with 500 ng of Nmrk2 luciferase constructs.

Cloning of Mouse Nmrk2 cDNA Into Recombinant Adenovirus

The cDNA of Nmrk2 was cloned by PCR amplification from reversed transcribed cardiac mRNA into pcDNA3HA vector, introducing a sequence with HA tag at the N-terminus of Nmrk2 open reading frame that replaced the first methionine. The HA tagged Nmrk2 cDNA was then subcloned into the pShuttle2 vector before to be inserted in the Adeno-X viral DNA (Clontech lab. Adeno-X Expression System 1). Pac1 Linearized recombinant adenoviral DNA was transfected in HEK 293 cells followed by rounds of infection to collect the adenoviral particles. Adenoviral titer was determined by the cytopathic effect method. NRCs were infected at day 3 of culture with 100 particles/cell.

Biochemical Studies

Frozen tissue samples were weighed, homogenized (Bertin Precellys 24) in ice-cold buffer (50 mg/ml) containing HEPES 5 mM (pH 8.7), EGTA 1 mM, DTT 1 mM and 0.1% Triton X-100. Citrate synthase (CS), activity was determined in homogenized ventricles as previously described (Kuznetsov et al. (2008). Analysis of mitochondrial function in situ in permeabilized muscle fibers, tissues and cells. Journal 3, 965-976). Activities of enzymes were determined by standard spectrophotometric assays. Briefly, for citrate synthase (CS), approximately 0.5 μg of protein were added in 1 ml of Buffer M (Trizma 100 mM (pH 8), 5,5′-dittiobis-(2-Nitrobenzenoic acid) 0.1 mM, Acetyl-CoA 0.3 mM, oxaloacetatic acid 0.5 mM) and the absorbance at 412 nm was measured during 3 min. Cytochrome oxidase (COX) activity was determined by the addition of approximately 0.25 μg of protein in 1 ml of phosphate buffer (K2HPO4 50 mM (pH 7.4)) containing 50 μM Cytochrome c (cytochrome c were previously reduced at 90% using sodium dithionite). Losing of reduced cytochrome c were followed by measuring the absorbance at 550 nm during 3 min using fully oxidized cytochrome c (by addition of potassium ferricyanure in excess) as a reference. For the determination of complexe I activity, 25 μg of protein and 100 μM NADH were added to 1 ml of C1 Buffer (41.1 mM KH2PO4, K2HPO4 8.8 mM, Decyl-ubiquinone 100 μM, BSA 3.75 mg/ml, pH 7.4). Measurements of the absorbance at 340 nm during 3 min in the presence and in the absence of 5 mM rotenone were used to calculate activity of this complexe. CS, COX and complexe I activities were respectively calculated using an extinction coefficient of 13600 M−1·cm−1, 18500 M−1·cm−1 and 6220 M−1·cm−1 and rates are given in UI/g prot.

Statistical Analyses

The data were expressed as mean±S.E.M. ANNOVA followed by post hoc Tukey test or unpaired Student's t test were used to determine the probability value (p value). A p value ≤0.05 was considered statistically significant. For in vivo experiments, animals were randomly assigned into different treatment groups.

### Results

The Nicotinamide Riboside Kinase 2 Pathway is Activated in the Failing Heart of SRF^{HKO} Mice and Nicotinamide Riboside Diet Protects Against Heart Failure

Transcriptomic analysis of the SRF^{HKO} hearts revealed that the Nmrk2 gene was induced as soon as day 8 (D8) after SRF inactivation and rose to 30-fold control levels at D25, the onset of DCM in this model (data not shown). Administration of phenylephrin (PE), an α-adrenergic agonist triggering cardiac hypertrophy in control and SRF^{HKO} mice as described previously (Tritsch et al. (2013). An SRF/miR-1 axis regulates NCX1 and Annexin A5 protein levels in the normal and failing heart. Journal 98, 372-380), further elevated Nmrk2 expression to 57-fold in SRF^{HKO} hearts versus 4-fold in control hearts (data not shown). The ectoenzyme NTSE (CD73) that hydrolyzes extracellular NAD⁺ and nicotinamide mononucleotide (NMN) to NR (Grozio et al. (2013). CD73 protein as a source of extracellular precursors for sustained NAD+ biosynthesis in FK866-treated tumor cells. Journal 288, 25938-25949), increased at D25 and to a greater degree upon PE treatment in SRF^{HKO} mice (data not shown). In parallel, Nampt, which converts NAM to NMN, and Pnp, which converts NR to NAM (Belenky et al. (2009). Nicotinamide Riboside and Nicotinic Acid Riboside Salvage in Fungi and Mammals: Quantitative Basis for Urh1 and Purine Nucleoside Phosphorylase Function in NAD+ Metabolism. Journal 284, 158-164; Belenky et al. (2007). Nicotinamide riboside promotes Sir2 silencing and extends lifespan via Nrk and Urh1/Pnp1/Meu1 pathways to NAD+. Journal 129, 473-484), were depressed upon PE treatment (data not shown). The expression of the redundant genes encoding NMNAT1, -2 and -3 enzymes, which convert NMN to NAD⁺ were not changed (data not shown). Genes involved in the de novo pathway originating from TRP and in the Preiss-Handler pathway originating from nicotinic acid (NA) were not modulated (data not shown).

Modulation of Nmrk2 and Nampt gene expression were corroborated by RT-qPCR analysis in a different series of mice than those used for transcriptomics (FIG. 3 a, b). Western blot analysis confirmed the robust induction of NMRK2 protein in the heart of SRF^{HKO} mice (data not shown). Immunofluorescent analysis of NMRK2 protein localization on frozen heart sections showed a low signal in the cytoplasm of cardiomyocytes and at the sarcolemma in control hearts (data not shown). The signal was strongly enhanced in SRF^{HKO} cardiomyocytes at D45.

Because NMRK2 is involved in NAD⁺ biosynthesis, we examined the possibility that Nmrk2 gene induction in the SRF^{HKO} heart may be a signature of altered NAD⁺ homeostasis.

There was a 30% loss of NAD⁺ levels at D15 in SRF^{HKO} hearts compared to controls (FIG. 3C). The gene expression pattern (Nt5e and Nmrk2 up with Pnp and Nampt down) suggested that cardiac tissue is attempting to mobilize and utilize NR as an NAD⁺ precursor while not increasing NAM availability or usage. We tested this hypothesis by intraperitoneal (i.p.) administration of NR and Nam to SRF^{HKO} mice from D8 to D15. Myocardial NAD⁺ levels were preserved by NR but not by NAM (FIG. 3c). Srf and Nmrk2 expression levels were not changed by these treatments (FIG. 3d, e). Because NR is orally available (Canto et al. (2012). The NAD(+) precursor nicotinamide riboside enhances oxidative metabolism and protects against high-fat diet- induced obesity. Journal 15, 838-847; Cerutti et al. (2014). NAD(+)-dependent activation of Sirt1 corrects the phenotype in a mouse model of mitochondrial disease. Journal 19, 1042-1049; Khan et al. (2014). Effective treatment of mitochondria' myopathy by nicotinamide riboside, a vitamin B3. Journal 6, 721-731; Trammell et al. (2016). Nicotinamide Riboside Opposes Type 2 Diabetes and Neuropathy in Mice. Journal 6, 26933; Zhang et al. (2016). NAD+ repletion improves mitochondrial and stem cell function and enhances life span in mice. Journal), we performed a second experiment in which we fed mice an NR-supplemented diet for 2 weeks from D5 to D20. We confirmed in this independent series that NAD⁺ levels are reduced in the SRF^{HKO} hearts compared to controls and can be rescued by NR administration (FIG. 3f).

NR Preserves Cardiac Functions in the SRF^{HKO} heart

Because NR protects myocardial NAD⁺, we hypothesized that NR supplementation in food might be beneficial for cardiac functions in the context of DCM-associated HF. We injected tamoxifen to control and mutant SRF^{HKO} mice from DI to D3 and started to feed the mice with NR-enriched food (0.22%) from D5 to D50 to reach a dose of 400 mg of NR/g of body weight/day. Mice fed with or without NR were sacrificed 3 days after the echocardiography at D50. Myocardial NAD, assayed by the colorimetric NAD recycling assay was decreased in the SRF^{HKO} mice at D50 (FIG. 3g) as at earlier stages (FIG. 3c, f). The NR-supplemented diet protected against the drop in NAD in SRF^{HKO} hearts. Weekly monitoring of body weight during this period showed that NR induced a modest 4 to 5% increase in body weight in controls and SRF^{HK)} mice (FIG. 4a). Cardiac parameters were analyzed by echocardiography between D45 to D47 when SRF^{HKO} mice develop HF (FIG. 4b-o). NR did not induce any change in heart rate and LV mass index (FIG. 4b, c). SRF^{HKO} mutant mice fed the standard diet displayed a severe decrease in LV ejection fraction (LVEF) and fractional shortening (FS) (FIG. 4d, e). The NR diet clearly protected against the decline in LV contractile functions in SRF^{HKO} mutant mice. LVEF was maintained at 64.8%±8.2 (S.D) versus 48.1% t 7.4 in the non-treated SRF^{HKO} mice (p=4.3×10⁻⁵) though it was still below the LVEF value of 73.5%±7.5 in control mice (p=3.8×10⁻²). The NR enriched-diet fully protected SRF^{HKO} mice against the dilatation of LV as measured in systole and diastole (FIG. 4g-i). The NR diet also reduced the thinning of the LV wall in systole and diastole, more efficiently at the level of the posterior wall than for the interventricular septum (FIG. 4j-m). This structural remodeling of the LV in the SRF^{HKO} translated into a reduction of the H/R ratio (mean LV wall thickness/LV radius) indicative of eccentric remodeling, which was fully prevented by NR (FIG. 4n). Interestingly, the NR diet slightly increased the H/R ratio in control mice, showing it can promote concentric remodeling, although it did not reach values associated with pathological cardiac hypertrophy, i.e. H/R>0.5 and increased LVMI. Changes in stroke volume (FIG. 4o) or cardiac output (not shown) were not significant.

The NR diet increased the expression level of the Nfel2 gene encoding NRF2 as well as its target gene metallothionein 2 (M12) in the heart of SRF^{HKO} mice (data not shown). NR also increased the expression level of the NADPH oxidase gene Nox4 (data not shown) but not Nox2 (not shown). The expression level of the glucose-6-phosphate dehydrogenase G6pdx initiating the pentose phosphate pathway, which is critical for NADPH production, was increased at baseline in SRF^{HKO} mice and maintained at higher level than in controls under the NR diet (data not shown). These results show that the SRF^{HKO} heart has transcriptional signs of ROS stress, that its NAD⁺ metabolome is depressed on account of low synthesis rather than high NAD⁺ turnover, and that, consistent with transcriptional induction of Arnirk2, its NAD⁺ can be corrected by NR, which also boosts antioxidant defenses.

NR Improves Metabolism of Citrate in HF

NR has been shown to enhance mitochondrial oxidative metabolism in liver and skeletal muscle tissues in response to high fat diet (Canto et al. (2012). The NAD(+) precursor nicotinamide riboside enhances oxidative metabolism and protects against high-fat diet-induced obesity. Journal 15, 838-847). HF might plausibly depress total mitochondrial biogenesis, thereby reducing production of phosphocreatine, ATP and ROS-detoxifying metabolites. Second, the electron transfer chain (ETC) might be damaged in HF, degrading production of high-energy phosphates. Third, expression and/or activity of particular enzymes might be depressed by HF and restored by NR.

Strikingly, citrate synthase activity was reduced to 65% control levels in the SRF^{HKO} left ventricular myocardium at D50 (FIG. 5). NR administration protected against the decline of citrate synthase activity in the failing heart.

Nmrk2 Gene is Induced by the AMPK-PPARα Axis

In terms of the requirement for ATP, PRPP and other energy inputs, the NR to NAD³⁰ pathway is the least expensive way for a cell to make NAD⁺ (de Figueiredo et al. (2011). Pathway analysis of NAD+ metabolism. Journal 439, 341-348). We hypothesized that Nmrk2 might be activated by pathways related to energy failure, a key step in the pathogenesis of HF. In agreement with this hypothesis, the level of phosphorylated AMPKα, the energy stress sensor AMP-activated kinase was increased at an early stage when Nmrk2 induction begins in the heart of SRF^{HKO} mice. The ratio of phosphorylated Acetyl-CoA Carboxylase (ACC) to total ACC, an indicator of AMPK activation, was also robustly increased (FIG. 6a). 5-Aminoimidazole-4-carboxamide ribonucleotide (AICAR) treatment stimulated AMPK phosphorylation in NRC and induced Nmrk2 expression at the protein level in NRC (FIG. 6b) to a level close to the effect of FK866 treatment. The energetic stress induced by glucose-deprivation for 24 h in NRC culture also tended to increase the expression of Nmrk2 though the impact on AMPK phosphorylation was not obvious.

AICAR treatment did not modulate NAD⁺ or NADH levels in NRC (data not shown) though it robustly induced Nmrk2 expression (FIG. 6c). To assess whether the effect of AICAR took place at the level of the Nmrk2 regulatory region, we transfected Nmrk2-luciferase constructs into NRC and treated them with AICAR. Analysis of the 5′ regulatory sequences of Nmrk2 revealed an enrichment in putative PPAR binding sites in the −3009 to −2205 region and in the −1028 to +61 region (FIG. 6d). To determine the contribution of these regulatory regions to the expression of Nmrk2 in cardiac cells, we performed a promoter deletion analysis. Maximal luciferase activity was obtained with the 3009 bp-long fragment. (FIG. 6d). Deleting 500 bp from the 5′ end reduced the activity by 60%. The region from −2552 to −-1028 did not contribute significantly to the activity but sequential deletions from −1028 to −228 progressively reduced luciferase activity

The p581-Luc was modestly responsive to AICAR treatment (FIG. 6e). On the other hand, the full-length p3009-Luc reporter was highly responsive to AICAR treatment (FIG. 6f). To assess whether activation of the Nmrk2 promoter by AICAR was depending on AMPK activation, we co-transfected a plasmid overexpressing a dominant negative (DN) isoform with the p3009-Luc construct (Woods et al. (2000). Characterization of the role of AMP-activated protein kinase in the regulation of glucose-activated gene expression using constitutively active and dominant negative forms of the kinase. Journal 20, 6704-6711). AMPK-DN efficiently blunted the induction of the p3009-Luc construct by AICAR (FIG. 6f).

To assess the role of PPAR factors in Nmrk2 gene regulation, we co-transfected NRC with the Nmrk2 p3009-Luc reporter and PPARα, PPARβ/δ or PPARγ expression vectors, together with the expression vector for their required partner, the retinoid X receptor. Among the 3 PPAR factors, only PPARα increased the activity of the Nmrk2 promoter in presence of its agonist GW7647 (FIG. 6g). The PPARα antagonist G6471 repressed the Nmrk2 promoter (FIG. 6g) and blunted the activating effect of AICAR (FIG. 6h). Neither the PPARβ antagonist GSK3787 nor the PPARγ antagonist G9662 had any effect on Nmrk2 promoter induction by AICAR. These results establish that Nmrk2 is an AMPK-PPAR α responsive gene that is induced by energy stress in cardiac cells. This effect is specific top cardiomyocytes since the activity of the Nmrk2 promoter was very low in cardiac fibroblasts compared to cardiomyocytes (FIG. 6i).

NR is Protective Against Cardiac Dysfunction and Remodeling Triggered by Pressure Overload

Our previous results showed that NR treatment is protective in a model of non-ischemic cardiomyopathy. To assess the impact of NR treatment on a different form of cardiac remodeling and heart failure, we chose the model of transverse aorta constriction (TAC) in mouse that simulate pressure overload-induced cardiac hypertrophy and heart failure as seen in patients suffering congenital bicuspid aortic valve or acquired aortic stenosis secondary to aortic valve lesion or calcification, or patients suffering hypertensive hypertrophic cardiomyopathy. We performed TAC surgery or SHAM surgery (control group, no aortic constriction). Each group of animal were divided in 2 subgroups fed either with regular chow diet (CD) or NR enriched diet at an average dose of 400 mg/kg/day for , with random assignation of each animal to each group. Our results show that NR treatment limits the drop in LVEF (FIG. 7a) and the thickening of the interventricular septum (IVSTh) (FIG. 7b) that is triggered by the TAC procedure compare to untreated TAC group. Quantification of myocardial NAD levels ((FIG. 7c-d-e) showed that NAD⁺ levels (FIG. 7c) are reduced at 6 weeks after TAC in both CD and NR groups compared to respective SHAM groups, but NAD⁺ levels remain significantly higher in NR treated TAC group than innon treated CD TAC group. NADH level and NAD+/NADH ration were not significantly affected by the TAC treatment (FIG. 7d-e). RT-qPCR analysis of cardiac mRNAs showed that the TAC procedure significantly increase Nmrk2 mRNA level ((FIG. 7f), has no effect on Nmrk1 mRNA level (FIG. 7g) and lower Nampt mRNA level ((FIG. 7h). NR treatment had no impact on the level of these mRNAs.

NMRK2 Protein is Increased when NAMPT Protein is Decreased in Human Failing Hearts

We analyzed NMRK2 and NAMPT protein levels in left ventricles from human healthy hearts as controls and failing hearts from patients diagnosed with non-obstructive cardiomyopathy. We observed a shift in the failing hearts toward decreased NAMPT expression and increased NMRK2, so a higher NMRK2/NAMPT ratio than in controls (FIG. 8). This shift is going in the same direction than observed in the mouse models at the mRNA level (FIG. 3A-B and FIG. 7f and h) and protein level (not shown). We also detected NMRK1 protein in human cardiac proteins extracts with comparable levels in healthy and failing hearts (data not shown).

### Discussion

We report findings that depressed NAD⁺ levels, lower expression of NAMPT and higher expression of Nmrk2 kinase are observed in mouse model of heart failure triggered by TAC or DCM and that administration of NR, the substrate of Nmrk2, prevents deterioration of cardiac functions and adverse remodeling. We also show that the same pattern of depressed NAMPT protein level and higher NMRK2 protein level is observed in human failing hearts suggesting that a shift from NAM recycling to NR-driven NAD⁺ biosynthesis is a common response of the heart to alterations in the energy systems and associated signaling. This observation further strengthens the idea that NR can be used as a drug able to sustain NAD+ biosynthesis in the human failing heart.

Altered NAD⁺ homeostasis and dysregulation of NAD⁺ biosynthetic and NAD⁺ consuming enzymes has been reported in several models of pressure overload or MI in recent years (Pillai et al. (2005). Poly(ADP-ribose) polymerase-1-dependent cardiac myocyte cell death during heart failure is mediated by NAD+ depletion and reduced Sir2alpha deacetylase activity. Journal 280, 43121-43130; Pillai et al. (2010). Exogenous NAD blocks cardiac hypertrophic response via activation of the SIRT3-LIKB1-AMP-activated kinase pathway. Journal 285, 3133-3144; Yamamoto et al. (2014). Nicotinamide mononucleotide, an intermediate of NAD+ synthesis, protects the heart from ischemia and reperfusion. Journal 9, e98972). In this context, much attention has focused on the Nampt enzyme that is repressed in several models of cardiac injuries (Hsu et al. (2014). The function of nicotinamide phosphoribosyltransferase in the heart. Journal 23, 64-68) as shown here in the TAC model of cardiac hypertrophy and heart failure and in the SRF^{HKO} model of DCM. However. whereas Nampt is depressed, there was a robust upregulation of Nmrk2 expression in the heart of SRF^{HKO} mice. Interrogation of the Gene Expression Omnibus (GEO) database reveals that Nmrk2 is induced in other models of DCM related to lamin-A mutation (GEO dataset GDS2746) (Muchir et al. (2007). Activation of MAPK pathways links LMNA mutations to cardiomyopathy in Emery-Dreifuss muscular dystrophy. Journal 117, 1282-1293) and isocitrate dehydrogenase 2 mutations (GEO dataset GDS4893) (Akbay et al. (2014). D-2-hydroxyglutarate produced by mutant IDH2 causes cardiomyopathy and neurodegeneration in mice. Journal 28, 479-490). Cardiac-specific PGC1α also leads to Nmrk2 upregulation (GEO dataset GDS4776) (Martin et al. (2014). A role for peroxisome proliferator-activated receptor gamma coactivator-1 in the control of mitochondrial dynamics during postnatal cardiac growth. Journal 114, 626-636). Interestingly, in parallel to induction of Nmrk2, there was an increase in the expression of Nt5e (CD73) that would promote conversion of extracellular NAD⁺ and NMN to NR, and a decline in the expression of Pnp which would convert NR into NAM (Belenky et al. (2007). Nicotinamide riboside promotes Sir2 silencing and extends lifespan via Nrk and Urh1/Pnp1/Meu1 pathways to NAD+. Journal 129, 473-484; Grozio et al. (2013). CD73 protein as a source of extracellular precursors for sustained NAD+ biosynthesis in FK866-treated tumor cells. Journal 288, 25938-25949) suggesting that the myocardium attempts to shift completely to an NR-driven pathway for NAD⁺ synthesis in the context of DCM. Nmrk1 is expressed at low level in the heart and was not modulated in any of these models. Baseline levels of Nmrk2 mRNA level appear to be high in normal human heart (e.g. GEO dataset: GDS426) in contrast to normal mouse heart (e.g. GEO dataset: GDS3142).

Our data show that Nmrk2 can be activated in response to Nampt repression and activation of the energy stress sensor AMPK. NMN synthesis from NR by Nmrk enzymes requires a single ATP while synthesis from NAM by Nampt requires more than 4 ATP equivalents: one for the autophosphorylation of the enzyme, and three (plus a carbohydrate) in formation of PRPP. Hence, the shift from Nampt to Nmrk2 for NAD⁺ synthesis is an energy-sparing mechanism that may be favored in HF.

We also found that Nmrk2 is an AMPK responsive gene. AMPK is activated in most models of HF, at least during the early steps of the disease, and is able to stimulate both glucose and fatty acid utilization to restore energy levels (Kim and Dyck (2015). Is AMPK the savior of the failing heart? Journal 26, 40-48). We observed an increased expression of the glucose transporter Glut1 and a downregulation of Pdk2 in the failing heart of SRF^{HKO} mice suggesting higher glycolytic flux while FAO related genes were repressed. Our analyses show that the stimulation of Nmrk2 expression by AMPK depends on PPARα activity. Though the latter is best known for its ability to stimulate FAO related genes, PPARα agonists have been shown to improve insulin sensitivity and glucose uptake in conjunction with AMPK activation in cardiac cells (Guerre-Millo et al. (2000). Peroxisome proliferator-activated receptor alpha activators improve insulin sensitivity and reduce adiposity. Journal 275, 16638-16642; Xiao et al. (2010). Peroxisome proliferator-activated receptors gamma and alpha agonists stimulate cardiac glucose uptake via activation of AMP-activated protein kinase. Journal 21, 621-626). In skeletal muscles, Nmrk2 has a higher expression level in muscles enriched in fast glycolytic fibers such as the plantaris than muscles enriched in slow oxidative fibers such as the soleus (not shown). Hence, activation of Nmrk2 expression in the failing heart may reflect the activation of glycolysis that is observed in many models of HF (Ventura-Clapier et al. (2010). Bioenergetics of the failing heart. Journal 1813, 1360-1372).

Although the Nmrk2 pathway is activated in the failing heart of SRF^{HKO} mice, the myocardial NAD⁺ level is depressed, which suggests that circulating and tissue levels of NAD⁺, NAM, NMN and NR are insufficient to sustain cardiac NAD⁺ synthesis in mice on a regular chow diet, stimulating an interest in NR supplementation to correct this defect. Several studies have shown that short-term NAD⁺ supplementation via osmotic pump delivery (14 days (Pillai et al. (2010). Exogenous NAD blocks cardiac hypertrophic response via activation of the SIRT3-LKB1-AMP-activated kinase pathway. Journal 285, 3133-3144) or

NMN supplementation by i.p. injection (3 days) (Karamanlidis et al. (2013). Mitochondrial complex I deficiency increases protein acetylation and accelerates heart failure. Journal 18, 239-250) can correct NAD⁺ deficiency in the heart. Two additional studies reported a beneficial impact of long-term NR supplementation in mouse models of mitochondrial (Cerutti et al. (2014). NAD(+)-dependent activation of Sirt1 corrects the phenotype in a mouse model of mitochondrial disease. Journal 19, 1042-1049; Khan et al. (2014). Effective treatment of mitochondrial myopathy by nicotinamide riboside, a vitamin B3. Journal 6, 721-731) and NR was originally shown to enhance oxidative metabolism in liver and muscle in the context of high-fat diet induced obesity (Canto et al. (2012). The NAD(+) precursor nicotinamide riboside enhances oxidative metabolism and protects against high-fat diet-induced obesity. Journal 15, 838-847).

We discovered a strongly beneficial effect of NR in this model of HF in preservation of cardiac functions and remodeling that was associated with maintenance of NAD⁺ levels in the heart. Experiments were conducted to probe the molecular nature of cardiomyocyte dysfunction in HF that is corrected by NR and to probe the basis for Nmrk2 induction in this model. The most striking NR-reversible deficit we found in HF was depression of citrate synthetase activity. Nmrk2 induction was shown to correlate with NAD⁺ decline and depend on the AMPK-PPARα axis.

Increasing the steady state level of NAD⁺ can be expected to increase activity of sirtuins (Brown et al. (2014). Activation of SIRT3 by the NAD+ precursos nicotinamide riboside protects from noise-induced hearing loss. Journal 20, 1059-1068; Canto et al. (2012). The NAD(+) precursor nicotinamide riboside enhances oxidative metabolism and protects against high-fat diet-induced obesity. Journal 15, 838-847; Gomes et al. (2013). Declining NAD(+) induces a pseudohypoxic state disrupting nuclear-mitochondrial communication during aging. Journal 155, 1624-1638). In addition, in the overfed mouse liver, the sum of NADP⁺ and NADPH was depressed and was brought back to normal by NR supplementation, suggesting that restoration of defense against ROS may mediate some of the protective effects of NR in obesity and type 2 diabetes (Trammell et al. (2016). Nicotinamide Riboside Opposes Type 2 Diabetes and Neuropathy in Mice. Journal 6, 26933). A progressive loss of Nampt expression, coupled with the known depression of ATP and high-energy phosphates, is postulated to make it challenging for damaged hearts to maintain NAD⁺ homeostasis. Consistent with in vitro data, depressed de novo synthesis of NAD⁺ may also reduce the steady state level of NAD⁺. AMPK was activated early in HF and, in addition to its well known roles in stimulating fuel oxidation to restore ATP levels (Kim and Dyck (2015). Is AMPK the savior of the failing heart? Journal 26, 40-48); this pathway results in cardiomyocyte induction of Nmrk2, which would allow synthesis of NAD⁺ in an ATP-conserving manner.

Many regulatory processes from gene expression to enzyme activity are controlled by reversible protein Lys acetylation. In the mitochondrial compartment, the degree of acetylation appears to be controlled by levels of Ac-coA and the activity of SIRT3, an NAD⁺-dependent protein lysine deacetylase (Ghanta et al. (2013). Mitochondrial protein acetylation as a cell-intrinsic, evolutionary driver of fat storage: chemical and metabolic logic of acetyl-lysine modifications. Journal 48, 561-574). In the nucleus and cytoplasm, though there are a variety of protein lysine acetyltransferases and families of deacetylases that are NAD⁺-dependent and NAD⁺-independent (Kouzarides (2000). Acetylation: a regulatory modification to rival phosphorylation? Journal 19, 1176-1179), production of citrate in mitochondria and its conversion to cytosolic Ac-coA is required to drive changes in histone acetylation (Wellen et al. (2009). ATP-citrate lyase links cellular metabolism to histone acetylation. Journal 324, 1076-1080). Our data are consistent with a HF-driven deficit in citrate production that would depress the supply of Ac-coA and that is restored by NR diet. Restoration of citrate synthase activity will also help improving the cardiac bioenergetics in the failing heart.

1. A method of treating cardiomyopathy in a subject in need thereof, comprising administering to the subject a therapeutically effective amount of nicotinamide riboside.

2. The method according to claim 1, wherein nicotinamide riboside is administrated in combination with a therapeutically effective amount of AMPK activator or/and a therapeutically effective amount of PPARα agonist.

3. The method according to claim 1, wherein nicotinamide riboside, the AMPK activator or/and the PPARα agonist are administered simultaneously, at essentially the same time, or sequentially.

4. The method according to claim 2, wherein the AMPK activator or/and the PPARα agonist is a small organic molecule.

5. The method according to claim 1, wherein cardiomyopathy is dilated cardiomyopathy.

6. The method according to claim 1, wherein nicotinamide riboside is administered orally.

7. The method according to claim 1, wherein nicotinamide riboside is administered as dietary supplement.

8. The method according to claim 1, wherein the cardiomyopathy is rare cardiomyopathy.

9. The method according to claim 8, wherein the rare cardiomyopathy is a symptom in a disorder selected from the group consisting of Congenital cardiomyopathy, Emery Dreiffuss Muscular Dystrophy, Duchenne and Becker Muscular Dystrophy, Limb-Girdle dystrophy, Steinert disease, Danon disease, Myofibrillar Myopathy, Arrhythmogenic dysplasia, Peripartum cardiomyopathy, Tako Tsubo cardiomyopathy, Nemaline Myopathies and RASopathies.

10. The method according to claim 8, wherein the rare cardiomyopathy derives from mutations or a rare variant of one or several genes selected from the group consisting of A2ML1, AARS2, ABCC9, ACAD9, ACADVL, ACTA1, ACTC1, ACTN2, AGK, AGL, AGPAT2, ALMS1, ANK2, ANKRD1, ANO5, ATP5E, ATPAF2, BAG3, BRAF, BSCL2, CALR3, CASQ2, CAV3, CAVIN4, CHRM2, COA5, COA6 , COL7A1, COQ2, COX15, COX6B1, CRYAB, CSRP3, CTNNA3, CTNNB1, DES, DLD, DMD, DNAJC19, DNM1L, DOL, DSC2, DSG2, DSP, DTNA, ELAC2, EMD, EYA4, FAH, FHL1, FHL2, FHOD3, FKRP, FKTN, FLNC, FOXD4, FOXRED1, FX, GAA, GATA4, GATA, GATA, GATAD, GFM1, GLA,GLB1, GNPTAB, GUSB, HCN4, HFE, HRAS, IDH2, ILK, JPH2, JUP, KCNH2, KCNJ2, KCNJ8, KCNQ1, KLF10, KRAS, LAMA2, LAMA4, LAMP2, LDB3, LIAS, LMNA, LZTR1, MAP2K1, MAP2K2, MIB1, MLYCD, MRPL3, MRPL44, MRPS22, MTO1, MYBPC3, MYH6, MYH7, MYL2, MYL3, MYLK2, MYOM1, MYOT, MYOZ2, MYPN, NEBL, NEXN, NF1, NKX2-5, NNT, NOTCH1, NRAS, OBSCN, OBSL1, OPA3, PDHA1, PDLIM3, PERP, PHKA1, PKP2, PKP4, PLN, PMM2, PPP1R13L, PRDM16, PRKAG2, PSEN1, PSEN2, PTPN11, RAF1, RASA2, RBM20, RITZ, RRAS, RYR2, SCN5A, SCO2, SDHA, SGCA, SGCB, SGC, SHOC, SLC22A5, SLC25A3, SLC25A4, SOS1, SOS2, SPEG, SPRED1, SURF1, SYNE1, SYNE2, TAZ, TBX20, TCAP, TGFB3, TMEM43, TMEM70, TMPO, TNNC1, TNNI3, TNNI3K, TNNT, TOR1AIP1, TPM1, TRIM63, TSFM, TTN, TTR, TXNRD2, VCL, and XK.

11. The method according to claim 8, wherein the rare cardiomyopathy derives from mutations or a rare variant of one or several genes selected from the group consisting of NAMPT, NMRK1, NMRK2, NMNAT, NMNAT2, NMNAT3, NADSYN1, NAPRT, TDO2, AFMID, KMO, KYNU, HAAO, QPRT, NT5E, SRF, MKL1, MKL2 and CKM.

12. The method according to claim 8, wherein the rare cardiomyopathy derives from mutations or a rare variant of one or several genes selected from the group consisting of PARP1, PARP2, CD38, BST1, and ART1.
